(19) United States
(12) Patent Application Publication
(10) Pub. No.: US 2023/0355649 A1
(43) Pub. Date: Nov. 9, 2023

(54) FORMULATIONS OF ANTI-VIRAL COMPOUNDS

(71) Applicant: EMPHASCIENCE, INC., Pittsford, NY (US)

(72) Inventors: Rebanta BANDYOPADHYAY, Webster, NY (US); Susen BANDYOPADHYAY, Webster, NY (US); Gurpartap SINGH, Princeton, NJ (US); Meghan M RODRIGUEZ, Victor, NY (US); Leann J VALENTINO, Rochester, NY (US)

(21) Appl. No.: 18/043,518

(22) PCT Filed: May 27, 2021
(86) PCT No.: PCT/US2021/070624

§ 371 Date: Feb. 28, 2023

Related U.S. Application Data
(60) Provisional application No. 63/071,560, filed on Aug. 28, 2020.
(60) Provisional application No. 63/200,334, filed on Mar. 2, 2021.

(51) Int. Cl. A61K 31/706; A61K 47/20; A61P 31/14

(52) U.S. Cl. CPC A61K 31/706; A61K 47/20; A61P 31/14

(57) ABSTRACT

A pharmaceutical composition comprising a compound of Formula (I)

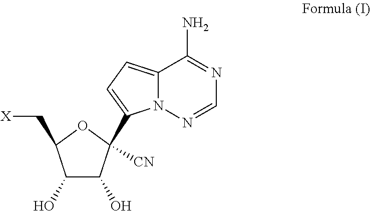

or a pharmaceutically acceptable salt thereof;
wherein X is selected from a hydroxyl, a metal salt hydroxylate, an O-linked phosphoester, an O-linked phosphoramidite, an O-linked ester, an O-linked carbamate, an S-linked phosphothioate, or an N-linked phosphoramidite, and
at least one pharmaceutically acceptable excipient selected from a cysteine compound, an amino acid, an N-acetyl amino acid, an acid or a salt thereof, or any combination thereof.

The pharmaceutical composition can be used for the effective treatment of viral infections in humans and other animal species caused by viruses, in particular, RNA viruses and can be administered orally or parenterally.

### TECHNICAL FIELD

The present disclosure relates to pharmaceutical compositions and methods of administration of antiviral drugs for the effective treatment of viral infections in humans and other animal species caused by viruses, in particular, RNA viruses. The antiviral drugs are nucleoside analogs, nucleotide analogs, prodrugs of nucleoside analogs, or prodrugs of nucleotide analogs that can inhibit genome replication of viruses.

### BACKGROUND

Viral infections can have a detrimental effect on life. Not only can there be an immediate impact on the health of an infected individual, the contagious nature of some viral infections can also have far reaching effects on the functioning of communities, businesses, services and the overall economy. This has been illustrated, for example, by the novel viral infection, COVID-19, which surfaced in 2019 resulting in a worldwide pandemic. COVID-19 presents the risk of severe respiratory failure and death in some patients. In many cases, the progression to acute symptoms occurs in older patients and in those with underlying medical conditions such as hypertension or diabetes.

Coronaviruses, in particular, are enveloped RNA viruses with a positive-sense, single-stranded RNA genome that infect both animals and humans. Diseases from coronavirus include the common cold, severe acute respiratory syndrome (SARS), Middle East respiratory syndrome (MERS), and severe acute respiratory syndrome by coronavirus 2 (SARS-CoV-2), the causative pathogen of the disease commonly known as COVID-19. The rapid emergence and spread of such viral infections allow little time for the development of vaccines. In the absence of clinically effective and safe vaccines, widespread immunization and controlling the virus becomes almost impossible. Therefore, alongside the development of vaccines, there is also a need for the effective treatment of viral diseases such as COVID-19.

Anti-viral compounds that mimic the structure of naturally occurring nucleosides and nucleotides constitute a significant part of our armament of drugs against viral infections. Nucleosides are part of the building blocks of DNA and RNA that are derived by the attachment of one sugar to one molecule of nucleobase, such as adenosine from nucleobase adenine and guanosine from guanine. Further addition of phosphate groups results in the formation of the so-called nucleotide. A nucleotide containing three phosphate groups, i.e. a triphosphate nucleoside is the form in which it exists in the RNA or DNA strand. Nucleoside analogs and nucleotide analogs compete with their natural counterparts to inhibit polymerase enzymes that help assemble the viral genome. These analogs can also act as chain terminators by being incorporated as defective building blocks and disrupting the chain of hydrogen bonds between nucleotides in a growing DNA or RNA strand. Such analogs are used as drugs against many serious viral diseases, including acquired immunodeficiency disease syndrome (AIDS), hepatitis, herpes and smallpox.

The monophosphate nucleotide prodrug Remdesivir (also known as GS-5734), has recently been approved for use against COVID-19, while also demonstrating anti-viral properties against a range of other viral infections. Remdesivir is a lipophilic adenosine monophosphate analog that is converted into the active triphosphate (GS-443902) inside the cell. While Remdesivir has recently shown efficacy in the treatment of viral infections such as COVID-19, current pharmaceutical compositions of Remdesivir have only been developed to be administered intravenously. The initial in vivo proof of concept of therapeutic activity was carried out against the Ebola virus in non-human primates by administering 10 mg/kg (body weight) of Remdesivir once a day by intravenous (IV) injection.

The currently marketed pharmaceutical compositions of Remdesivir are formulated either as a lyophilized powder or a concentrated, ready-to-dilute solution of Remdesivir in water for injection. Both formulations include very high amounts sulfobutylether β-cyclodextrin (SBEβCD) at up to 30 to 60 times the weight of Remdesivir dose as a complexing and solubilizing excipient. Hence, up to 6 g of SBEβCD can be administered along with a 100 mg unit dose of Remdesivir by intravenous infusion. This is disadvantageous because following intravenous administration, cyclodextrins are excreted intact by renal excretion and can accumulate in the kidney at high doses. This can cause vacuolation of the tubular cells in the kidney leading to possible renal impairment. In children, and in patients with lower glomerular filtration rate due to impaired kidneys, high doses of SBEβCD can result in osmotic nephrosis and extra renal adverse effects due to higher blood levels of SBEβCD and increase in osmotic pressure. There is therefore a need to develop pharmaceutical compositions that are free of cyclodextrins, while still ensuring that nucleoside analogs, such as Remdesivir, are still effectively solubilized.

Furthermore, prior art compositions of nucleoside analogs and nucleotide analogs including Remdesivir are considered unviable for different routes of administration due to a number of factors including their limited solubility. For oral administration, prior art compositions comprising Remdesivir are anticipated to have poor absorption in the stomach and gut, instability in the intestinal media and membranes, in the blood and due to hepatic first-pass metabolism (such as in the presence of many CYP and carboxylase enzymes), risk of accumulation and toxicity in the liver and, as a result of all these, low bioavailability.

It was therefore an aim of the present inventors to develop improved pharmaceutical compositions and methods of administering nucleoside analogs and nucleotide analogs such as Remdesivir that have improved bioavailability. It was also an aim of the present inventors to develop pharmaceutical compositions that can be used for various modes of administration, including oral administration and/or administration by injection.

### SUMMARY OF INVENTION

In a first aspect, there is provided a pharmaceutical composition comprising a compound of Formula (I)

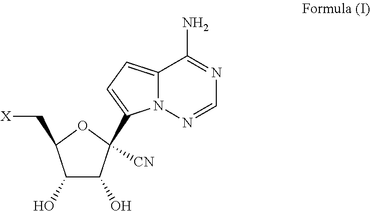

or a pharmaceutically acceptable salt thereof;
wherein X is selected from a hydroxyl, a metal salt hydroxylate, an O-linked phosphoester, an O-linked phosphoramidite, an O-linked ester, an O-linked carbamate, and S-linked phosphothioate, or an N-linked phosphoramidite, and at least one pharmaceutically acceptable excipient selected from a cysteine compound, an amino acid, an amino acid salt, an N-acetyl amino acid, an acid or salts thereof or any combination thereof. The amino acid salt may be a hydrochloride salt. Such pharmaceutically acceptable excipients are widely considered safe and well tolerated. In some embodiments, the at least one pharmaceutically acceptable excipient is an acidulant or a pH adjusting agent that is used to reduce the pH of the solution. In some embodiments, the acid is an organic acid.

In some embodiments, the at least one pharmaceutically acceptable excipient comprises at least one cysteine compound. As shown in the Examples, pharmaceutical compositions comprising at least one cysteine compound show improved solubilization of anti-viral compounds in accordance with Formula (I), with certain cysteine compounds showing improved solubilization compared to sulfobutylether beta-cyclodextrin (SBEβCD). In some embodiments, the % w/w ratio of the at least one cysteine compound to the compound of Formula (I) is at least 1:1, or optionally equal to or greater than 1.5:1. In some embodiments, the at least one cysteine compound comprises cysteine, glutathione (i.e., a cysteine containing dipeptide), cysteine hydrochloride, N-acetyl-cysteine or a combination thereof. In some embodiments, the at least one cysteine compound comprises cysteine hydrochloride and/or N-acetyl-cysteine. In some embodiments the at least one cysteine compound comprises cysteine hydrochloride and N-acetyl-cysteine. In some embodiments, the at least one cysteine compound may be an acidulant or a pH adjusting agent used to lower the pH of the solution.

In some embodiments, the at least one pharmaceutically acceptable excipient comprises at least one acid or salt thereof. The acid may act as an acidulant. In some examples the acid may be an organic acid. The at least one organic acid or salt thereof may be selected from lactic acid, acetic acid, citric acid, formic acid, oxalic acid, ascorbic acid, uric acid, malic acid, tartaric acid or any combination thereof. Organic acids and salts thereof can in some examples improve the solubilization of anti-viral compounds, such as Remdesivir. In some embodiments, the pharmaceutical composition comprises a combination of at least one acid or salt thereof and at least one cysteine compound. In some embodiments, the acid is an organic acid.

In some embodiments, the at least one pharmaceutically acceptable excipient comprises at least one N-acetyl amino acid. In some embodiments, the at least one N-acetyl amino acid is N-acetyl alanine or N-acetyl cysteine. In some embodiments, the at least one pharmaceutically acceptable excipient comprises an amino acid hydrochloride. In some embodiments, the pharmaceutical composition comprises at least one amino acid hydrochloride and at least one cysteine compound, or at least one amino acid hydrochloride and at least one N-acetyl amino acid.

The at least one pharmaceutical excipient may improve the dissolution and solubilization of the compound of Formula (I) in aqueous solution, and may have improved bioavailability as compared to prior art pharmaceutical compositions. As a result, the pharmaceutical compositions described herein may allow for an adequate unit dose to be delivered to a patient for effective treatment without increasing the risk of toxicity and adverse effects, in contrast to the challenges of poor bioavailability of anti-viral compounds, such as Remdesivir.

In some embodiments, the pharmaceutical composition is formulated such that the compound of Formula (I) has a solubility of greater than 0.05 mg/mL when placed in an aqueous solution at pH 3 to 7, or at pH 4 to 6.5, or pH 4.5 to 6.0, or pH 5 to 5.5. This may ensure that the compound of Formula (I) can remain in solution when administered as an injectable solution within an acceptable pH range, and can also be readily absorbed from the small intestine and generally from the gastrointestinal tract following oral administration.

In some embodiments, the pharmaceutical compositions described herein, unlike previous Remdesivir compositions, are free of cyclodextrins (for example, free of sulfobutylether cyclodextrin) that could interfere with other biochemical processes in the patient's body. In certain cases, cyclodextrins are also known to accumulate in the kidney, causing renal impairment.

In some embodiments, the pharmaceutical composition is a liquid formulation. Liquid formulations can be advantageous because the compound of Formula (I) is pre-solubilized. In some examples, the liquid formulation described herein may be administered directly, or can be formed by dilution. In both cases, a liquid formulation is advantageous since this avoids additional compounding steps and reconstitution of the powder, which can be both time-consuming, expensive and may increase the chance of inaccurate dosing. The risks of inadvertent exposure to the preparer may also be significantly reduced.

In some embodiments the pharmaceutical composition is a solution, a suspension or a mixture thereof.

In some embodiments, the pH of the liquid formulation is less than 8.5, optionally wherein the pH of the liquid formulation is from 1-<8. As demonstrated in the application Examples, an acidic pH and/or the presence of an acidulant may improve the solubilization of the compound of Formula (I).

In some embodiments, the pharmaceutical composition comprises one or more co-solvents. In some embodiments, the one or more co-solvents may aid the solubilization of the compound of Formula (I). In some embodiments, the one or more co-solvents may reduce precipitation of the compound when the pharmaceutical composition is added to an aqueous solution. In some embodiments, the pharmaceutical composition comprises one or more co-solvents in combination with a compound of Formula (I) and one or more pharmaceutical excipients selected from cysteine compounds, amino acid hydrochlorides, N-acetyl amino acids, or inorganic and organic acids. In some embodiments, the one or more co-solvents comprises low molecular weight polyethylene glycols (PEG), propylene glycol, benzyl alcohol, ethanol or a combination thereof.

In some embodiments, the pharmaceutical composition comprises PEG. In some embodiments, PEG is present in an amount >40% w/w of the pharmaceutical composition. In some embodiments, PEG has a molecular weight from 200 to 1000. In some embodiments, PEG has a molecular weight from 200 to 600. In some embodiments, PEG has a molecular weight of 300 and/or 400. In some embodiments, the pharmaceutical composition comprises a combination of PEG and benzyl alcohol. In some embodiments, the pharmaceutical composition comprises a combination of PEG and ethanol. In some embodiments, the pharmaceutical composition comprises a combination of PEG, benzyl alcohol and ethanol. In some embodiments, the one or more co-solvents is free of ethanol. This may improve the stability of the pharmaceutical composition, e.g., the stability of the compound of Formula (I), such as Remdesivir.

In some embodiments, the pharmaceutical composition comprises one or more surfactants. In some embodiments, the one or more surfactants may comprise a polysorbate or a polyoxy n castor oil, wherein n is 30 to 40, or a block copolymer of poly(ethylene oxide) (PEO) and poly(propylene oxide) (PPO), such as, poloxamer (e.g. poloxamer 188) or Pluronic®. In some embodiments, the one or more surfactants may comprise polysorbate 20, 40, 60, 80, polyoxyl 35 castor oil or a combination thereof. In some embodiments, the one or more surfactants is selected from polysorbate 20, polysorbate 40, polysorbate 60, polysorbate 80, polyoxyl 35 castor oil, cremophor, polyoxyethylene (20) sorbitan monooleate, polyethylene glycol sorbitan monooleate, polyoxyethylenesorbitan monooleate, or a block copolymer of poly(ethylene oxide) (PEO) and poly(propylene oxide) (PPO), such as, poloxamer (e.g. poloxamer 188), or a combination thereof. In some embodiments, the pharmaceutical composition comprises a polysorbate (e.g. polysorbate 80) in combination with a triblock copolymer (e.g. a poloxamer, such as poloxamer 188). In some embodiments, the one or more surfactants (or at least one of the one or more surfactants) has a HLB value from 10-20, optionally from 12 to 18, or optionally from 14 to 16, optionally about 15. In some embodiments, the one or more surfactants may comprise polysorbate, for example, polysorbate 80. In some embodiments, the pharmaceutical composition comprises two surfactants. In some embodiments, the pharmaceutical composition comprises a first surfactant with a HLB value from 10-20, optionally from 12 to 18, or optionally from 14 to 16, optionally about 15, and a second surfactant with a HLB value greater than 20, for example, about 25 to 35. In some embodiments, the one or more surfactants may demonstrate improved solubilization of the compound of Formula (I) and/or the other excipients, and/or reduce precipitation or phase separation of any of the constituents of the formulation when the pharmaceutical composition is added to an aqueous solution. In some embodiments, the pharmaceutical composition comprises one or more surfactants in combination with a compound of Formula (I) and one or more pharmaceutical excipients selected from cysteine compounds, amino acid hydrochlorides, N-acetyl amino acids or acids. In some embodiments, the acids are organic acids. In some embodiments, the pharmaceutical composition comprises one or more surfactants in combination with one or more co-solvents, a compound of Formula (I) and one or more pharmaceutical excipients selected from cysteine compounds, amino acid hydrochlorides, N-acetyl amino acids, inorganic acids or organic acids.

In some embodiments, the pharmaceutical compositions described herein are compatible with various modes of administration. Accordingly, the present disclosure provides pharmaceutical compositions that have increased solubility of the compounds of Formula (I) compared with known pharmaceutical compositions. The present disclosure also may provide pharmaceutical compositions with increased stability (e.g. in solution) of the compounds of Formula (I) compared with reconstituted solution comprising Remdesivir used for IV administration. These pharmaceutical compositions may be administered by various different methods, including oral administration and parenteral administration by injection, or by inhalation, nebulisation, intratracheal instillation or nasal administration. In some embodiments, the pharmaceutical composition is an oral formulation or a parenteral formulation.

In some embodiments, the pharmaceutical composition is an oral formulation and the method of administration is oral administration. Oral administration may be less invasive than other forms of administration. As a result, oral formulations may be easily self-administered by a patient without assistance from a medical professional. Oral formulations can therefore be used more widely and to treat many more infected individuals, including patients that do not have access to hospitals or do not require admittance to a hospital for such treatment. Such oral formulations can also complement and extend the therapy to patients by switching them from an intravenous administration method and formulation in the hospital and to allow them to continue therapy even after discharge from the hospital to reduce cost and risk of acquiring other infections in the hospital for more vulnerable patients, such as, for individuals who have underlying chronic diseases or are immunocompromised. For pandemics, such as, COVID-19, the availability of an oral formulation and dosage form and the option to be treated at home or away from the hospital, would reduce risk of disease transmission and open up hospital beds for critically ill patients. With oral administration, treatment can be initiated at the discretion of a healthcare provider even when the patients are at their own homes and early in the disease progression, when symptoms may still be mild and manageable without hospitalization.

In some embodiments, the pharmaceutical composition is an injectable solution and the method of administration is by injection. In some embodiments, the injection is an intravenous injection or a subcutaneous injection. As compared to prior art intravenous pharmaceutical compositions, the pharmaceutical compositions of the present application may more effectively solubilize the compound of Formula (I) as compared to prior art compositions. This may be advantageous for the preparation of injectable solutions with reduced risk of adverse effects and may also lead to more accurate dosing.

In some embodiments, the pharmaceutical compositions may further comprise one or more further additional pharmaceutically acceptable excipients (i.e. in addition to at least one pharmaceutical excipient selected from cysteine compounds, amino acid hydrochlorides, N-acetyl amino acids, an acid or salt thereof).

The pharmaceutical excipients described herein may improve the physical and chemical stability of the compound of Formula (I) as compared to previous compositions. Pharmaceutical excipients described herein may additionally protect and/or improve the shelf-life of a product compound of Formula (I). Any pharmaceutical excipient described herein (e.g. complexing agents, polymers, surfactants, metal salts, chelating agents, anti-oxidants, phospholipids and other amphoteric molecules to form liposomes and micelles or a combination thereof, in addition to at least one pharmaceutical excipient selected from cysteine compounds, amino acid hydrochlorides, N-acetyl amino acids, an acid or salt thereof) may additionally protect the product comprising the compound of Formula (I) from being chemically degraded and/or improve intestinal and cellular uptake of the compound of Formula (I).

In some embodiments, the pharmaceutical compositions have improved shelf-stability as compared with known compositions of anti-viral compounds, such as Remdesivir.

In some embodiments and examples, the metabolised active product of the compound of Formula (I) is Compound (D)

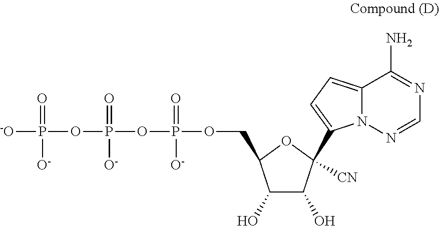

In some embodiments and examples, the compound of Formula (I) is

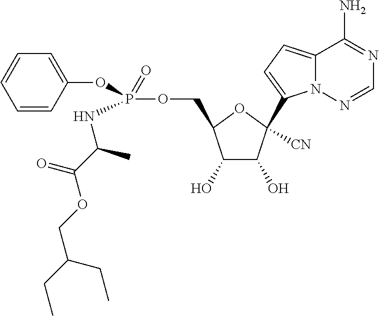 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

This compound is otherwise known as Remdesivir. Remdesivir has good anti-viral properties against SARS-CoV-2 and other viral infections.

In alternative embodiments, the compound of Formula (I) is

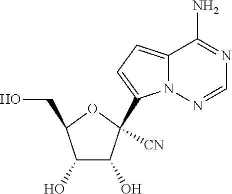 [SMILES: N#C[C@@]1(c2ccc3c(N)ncnn23)O[C@H](CO)[C@@H](O)[C@H]1O]

or a salt form thereof. This is the free nucleoside variant (GS-441524) of Remdesivir, but can be phosphorylated to the active triphosphate (i.e. Compound D) in the cell.

In other embodiments, the pharmaceutical composition comprises a compound of Formula (I) that is a chemically modified analog or a natural metabolite of Remdesivir. Chemical modifications over Remdesivir may include one or more of the following 1) using different O-linked phosphoramidites including modified forms of the alanine metabolite; 2) using O-linked phosphoramidites having a different stereochemistry than Remdesivir, 3) using analogues such as O-linked esters, O-linked carbamates, or the hydroxylate salt. These can be metabolised to the free nucleoside (GS-441524) in the cell, and in some instances can have improved solubility and/or stability as compared with Remdesivir; 4) using salt forms of compounds of Formula (I); and 5) using compounds of Formula (I) that have reduced log P and/or increased log S, where ‘P’ represents octanol-water partition coefficient of the compounds of Formula (I), which is a measure of liphophilicity and ‘S’ represents solubility, as compared with Remdesivir.

### Method of Treating

In a second aspect, there is provided a method of treating a viral infection, the method comprising administering to a subject in need thereof a therapeutically effective amount of the pharmaceutical compositions described herein. In some embodiments, the viral infection is an RNA viral infection. In some embodiments, the virus causing the viral infection is selected from a coronavirus, respiratory syncytial virus, ebola, hepatitis, junin, lassa fever, orthomyxovirus, Hepatitis Virus (HV) type, disease-causing picornavirus, Ebola, SARS, MERS, respiratory syncytial virus and other pneumovirus, influenza, polio measles and retrovirus including adult Human T-cell lymphotropic virus type 1 (HTLV-1) and human immunodeficiency virus (HIV). In some embodiments, the viral infection is a coronavirus infection. In some embodiments, the viral infection is SARS-CoV-2.

In some embodiments, the pharmaceutical composition is administered orally. In other embodiments, the pharmaceutical composition is administered by injection. In some embodiments, the pharmaceutical composition is administered by inhalation, nebulisation, intratracheal instillation or nasal administration. In some embodiments, the injection is an intravenous injection or a subcutaneous injection.

In some embodiments, the pharmaceutical composition for use in a method of treating viral infection is a liquid formulation. In some embodiments, the amount of liquid formulation administered orally is from about 1 mL to about 6 mL of liquid formulation. In some embodiments, the amount of liquid formulation administered by intravenous infusion is from about 100 mL to about 250 mL following the dilution of 1 to 40 mL of the liquid formulation containing a higher concentration of the active ingredient than the final infusate.

In some embodiments of the pharmaceutical composition for use in a method of treating viral infection, the amount of compound of Formula (I) administered is from 20 mg to 300 mg, or from 50 mg to 250 mg, or from 100 mg to 200 mg. In some embodiments, the amount of compound of formula (I) administered is greater than 20 mg, or greater than 50 mg, or greater than 75 mg, or greater than 90 mg. In some embodiments, the amount of compound of formula (I) administered is less than 180 mg, or less than 160 mg, or less than 140 mg, or less than 120 mg.

### Capsule(s), Oral Solution(s) and Injectable Solution(s)

In a third aspect, there is provided a capsule comprising the pharmaceutical composition as described herein. In an embodiment, the capsule is a liquid-fill capsule. In some embodiments, the liquid fill capsule has a volume from about 0.4 mL to about 0.9 mL, optionally from about 0.6 mL to about 0.8 mL, optionally about 0.7 mL (i.e. wherein the volume corresponds to the amount of liquid formulation in the capsule).

In a fourth aspect, there is provided an oral solution comprising the pharmaceutical composition as described herein. The oral solution may be in the form of a medicine, a syrup, an elixir, syrup or a suspension.

In a fifth aspect, there is provided an injectable solution comprising the pharmaceutical composition as described herein.

The details, examples and preferences provided in relation to any one or more of the stated aspects of the present invention will be further described herein and apply equally to all aspects of the present invention. Any combination of the embodiments and preferences described herein in all possible variations thereof is encompassed by the present invention unless otherwise indicated herein, or otherwise clearly contradicted by context.

### BRIEF DESCRIPTION OF FIGURES

FIG. 1: shows the stability of Remdesivir in solution at different pH, determined by the % degradation of the compound per hour at 40° C.

FIG. 2: shows the % dissolution of active compounds in aqueous solution over time. The diamond data points correspond to an exemplary pharmaceutical composition of the invention (open diamond, dashed line—without capsule, filled diamond, solid line—filled into a hard-gelatin capsule). The circle data points with thin dashed line correspond to a comparative control comprising a powdered drug with high bioavailability, acetaminophen, filled into hard-gelatin capsule. The square data points represent the dissolution of Remdesivir drug substance as a powder filled into hard-gelatin capsule either with a solubilizing excipient, SBEβCD (open squares) or without any other excipient (closed squares).

FIG. 3: shows the mean plasma profile of the Remdesivir metabolite Compound (C), GS-441524, in Male Beagle Dogs following either a 30 Minute IV Infusion (lighter line) or a Single PO Dose of 20 mg/kg Remdesivir (darker line).

### DETAILED DESCRIPTION

When ranges are used herein, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range may vary. Typical experimental variabilities may stem from, for example, changes and adjustments necessary during scale-up from laboratory experimental and manufacturing settings to large scale, GMP manufacturing conditions as is known to those familiar with the art of pharmaceutical development and manufacturing. Such changes can vary between 1% and 10% of the stated number or numerical range.

The term “prodrug” refers to a molecule that may or may not have pharmacological activity on its own, but is chemically altered within the subject's body after administration either due to metabolism or due to exposure to a physiological medium or from biochemical processes in the cell, or otherwise to produce the pharmacologically active drug after administration. In the context of this disclosure, the pharmacologically active drug is a molecule that is able to inhibit, block or stall the replication of viral genome. In some embodiments, the pharmacologically active drug is a triphosphate.

The term “comprising” (and related terms such as “comprise” or “comprises” or “having” or “including”) has an open meaning and therefore a pharmaceutical composition comprising described features may comprise additional components in addition to the described features. The term “comprising” (and related terms) also may include those embodiments, for example, an embodiment of any composition of matter, composition, method, or process, or the like, that “consist of” or “consist essentially of” the described features, i.e., are limited to the described features.

Abbreviations used herein have their conventional meaning within the chemical and biological arts, unless otherwise indicated.

The term “oral formulation” is a finished dosage form and composition thereof, which is administered by mouth by an act of ingestion. Oral formulation is taken to exclude intravenously administered formulations, or any dosage form and composition that can be injected, or inhaled or administered by other routes of administration, such as, rectal, topical or transdermal.

The term ‘HLB value’ is the hydrophilic lipophilic balance of a surfactant. The HLB value is a measure of how hydrophilic or lipophilic a surfactant is, with HLB numbers >10 have an affinity for water (hydrophilic) and number <10 have an affinity of oil (lipophilic). For non-ionic surfactants the HLB value is determined by the Griffin's method wherein HLB=20*Mₕ/M where Mₕ is the mass of the hydrophilic components, and M is the mass of the whole molecule. HLB values for ionic surfactants (i.e. wherein HLB values >20) can be determined using the Davies method.

The term “acidulant” refers to a compound that is an acidifying agent, i.e., an agent that lowers the pH of a composition or formulation.

The term “effective amount” or “therapeutically effective amount” refers to the amount of compound of Formula (I) or metabolites thereof described herein that have adequate antiviral activity needed to bring about an acceptable outcome of the therapy as determined by the lessening of severity of the disease and/or complete remission of the disease as measurable by clinical, biochemical or other indicators that are familiar to those trained in the art. The therapeutically effective amount may vary depending upon the intended application (in vitro or in vivo), or the subject and the viral disease being treated, as well as, the disease condition being treated, e.g., the weight and age of the subject, the severity of the disease condition, the manner of administration and the like, which can readily be determined by one of ordinary skill in the art. The term also applies to a dose that will induce a particular response in target cells. The specific dose will vary depending on the particular compound of Formula (I) (i.e. compound) chosen, the dosing regimen to be followed, whether it is administered in combination with other compounds, route and timing of administration, the tissue to which it is administered, and the physical delivery system in which it is carried.

The terms “treatment” and “treating” refer to an approach for obtaining beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

The term “therapeutic effect,” as that term is used herein encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “subject” or “patient” refers to an animal, such as a mammal, for example a human. The methods described herein can be useful in both human therapeutics and veterinary applications. In some embodiments, the patient is a mammal, and in preferred embodiments, the patient is human. For veterinary purposes, the term “subject” and “patient” include, but are not limited to, farm animals including cows, sheep, pigs, horses, and goats; companion animals such as dogs and cats; exotic and/or zoo animals; laboratory animals including mice, rats, rabbits, guinea pigs, and hamsters; and poultry such as chickens, turkeys, ducks, and geese.

The term “alkyl” refers to any substituted or unsubstituted alkane missing one hydrogen. In some embodiments, C₁-C₆ alkyl may include methyl, ethyl, n-propyl, i-propyl, n-butyl, s-butyl, i-butyl, t-butyl, n-pentyl, i-pentyl, s-pentyl, t-pentyl, neopentyl, 3-pentyl, secisopentyl or 2-methylbutyl. In some examples, C₁-C₆ alkyl is taken to include C₁-C₆haloalkyl, e.g., wherein the alkyl comprises at least one halo-substituent selected from fluoro, chloro, bromo or iodo.

The term “allyl” refers to any substituted or unsubstituted alkene missing one hydrogen. In some embodiments, C₂-C₆ alkenyl may include ethylene, propylene, butylene or pentylene. In some examples, C₂-C₆ allyl is taken to include C₂-C₆ haloallyl e.g., wherein the allyl comprises at least one halo-substituent selected from fluoro, chloro, bromo or iodo.

The term “alkynyl” refers to any substituted or unsubstituted alkyne missing one hydrogen. In some embodiments, C₂-C₆ alkynyl may include acetylyne, propylyne or butylene. In some examples, C₂-C₆ alkynyl is taken to include C₂-C₆ halo alkynyl e.g., wherein the alkynyl comprises at least one halo-substituent selected from fluoro, chloro, bromo or iodo.

The term “phenyl” may relate to any unsubstituted phenyl or substituted phenyl. The substituted phenyl may be substituted with one or more substituents selected from: fluoro, chloro, bromo, iodo, methoxy, ethoxy, nitrile, amino, hydroxyl, C₁-C₆ alkyl, C₂-C₆ allyl, C₂-C₆ alkynyl, C₁-C₆ alkoxyl.

The term “biphenyl” is taken to include an unsubstituted biphenyl and a biphenyl group substituted with one or more of: fluoro, chloro, bromo, iodo, methoxy, ethoxy, nitrile, amino, hydroxyl, C₁-C₆ alkyl, C₂-C₆ allyl, C₂-C₆ alkynyl, C₁-C₆ alkoxyl.

The term “alkoxy” refers to a group having the formula O-alkyl, wherein the alkyl group is as defined above.

The term “amino” refers to any nitrogen radical having the formula NX₂, wherein X is either H, or alkyl; wherein alkyl is as defined above.

The term “heteroaryl” refers to an aromatic group comprising at least one of O, N or S. In some embodiments, the heteroaryl is pyridine, bipyridine, furan, indole, pyrrole, thiazole, thiophene, imidazole, oxazole, thiazole or furazan. “Heteroaryl” is taken to include an unsubstituted heteroaryl and a heteroaryl that is substituted with one or more of fluoro, chloro, bromo, iodo, methoxy, ethoxy, nitrile, amino, nitro, cyano, azido, hydroxyl, C₁-C₆ alkyl, alkoxy, C₂-C₆ allyl, C₂-C₆ alkynyl, C₁-C₆ alkoxy.

The term “peptide” refers to a portion of a molecule comprising at least two amino acids that are linked via an amide bond.

The term “O-linked ester” refers to an ester that is attached via an O atom.

The term “phosphoramidite” is used to describe amides phosphoric acid that are commonly employed in the synthesis of differentially protected phosphate esters as prodrugs. In addition, they can be useful for the synthesis of phosphate monoesters by hydrolysis of the phosphoramide bond

The term “O-linked phosphoramidite” refers to any phosphoramidite (e.g. phosphate linked to N) that is attached via an O atom.

The term “S-linked phosphothioate” refers to any phosphothioate (e.g. phosphate linked to S) that is attached via an S atom.

The term “N-linked phosphoramidite” refers to any phosphoramidite (e.g. phosphate linked to N) that is attached via an N atom.

The term “O-linked phosphoester” refers to any phosphoester that is attached via an O atom.

The term “O-linked carbamate” refers to any carbamate that is attached via an O atom.

The term “log P” refers to the log of the partition coefficient “P”, which is defined as the ratio of the concentration of an unionized solute between octanol and water. In some examples, the log P may be determined by chemical software, e.g., by ALOGPS 2.1.

$$log {P}_{oct / wat} = log ( \frac{{[ solute ]}_{octanol}^{un - ionized}}{{[ solute ]}_{water}^{un - ionized}} ) .$$

Alternatively, the term “log D” may be used to describe the Log P of the ionized moiety in water at a given pH, where “D”, is defined as the distribution coefficient and expressed as the ratio of the concentration of the ionized solute between octanol and water at said pH.

The term “log S” refers to the water solubility of a drug and is defined as a common solubility unit corresponding to a 10-based logarithm of the solubility of a molecule measured in mol/L under standard conditions. In some examples, the log S may be determined by chemical software, e.g., by ALOGPS 2.1.

The term “bioavailability” generally describes the extent to which a given dose of a drug or metabolite thereof enters systemic circulation and thereby is available to produce a pharmacological effect. Bioavailability can also be measured by quantifying the AUC, wherein “AUC” refers to the area under the plasma-concentration and time curve which describes the variation of a drug concentration in blood plasma as a concentration of time and expressing it as a fraction of the AUC that would result if the entire dose were to be available in the systemic circulation. For the purpose of quantifying bioavailability, the AUC from all other routes of administration may be divided by the AUC obtained from intravenous route of administration with the assumption that the drug is 100% bioavailable following intravenous administration and that the AUC from the intravenous administration represents the maximum value for a given dose. The plasma concentrations for the estimation of AUC can be determined by various analytical methods including liquid chromatography linked to mass spectrometry (LC/MS), or gas chromatography linked to mass spectrometry (GC/MS) radiolabelling, etc. Bioavailability may be represented as a fraction or, more commonly, as the percentage of a therapeutically active drug that reaches the systemic circulation compared to via intravenous administration. By way of example, for oral administration, determined by (AUCₒᵣₐₗ/AUCᵢₙₜᵣₐᵥₑₙₒᵤₛ)×100.

The term Cₘₐₓ is the maximum concentration in the blood that is achieved following administration of the drug.

Any method of treating a disease described herein, e.g., the method comprising administering to a subject in need thereof a therapeutically effective amount of the pharmaceutical composition described herein may be rephrased or reformulated as “a medicament for use in the treatment of a disease”. In other words, if the disease is a certain type of viral infection the method of treatment can be rephrased to “a pharmaceutical composition for use in the treatment of a viral infection”.

Any structure or formula disclosed herein which are demonstrated in ionized form, can also include the non-ionized variants.

### Pharmaceutical Composition

### Pharmaceutical Excipient

The pharmaceutical composition may comprise at least one pharmaceutically acceptable excipient selected from a cysteine compound, an amino acid hydrochloride, an N-acetyl amino acid, an acid or a salt thereof, or any combination thereof. In some embodiments, the at least one pharmaceutically excipient is an acidulant.

### Cysteine Compound

In some embodiments, the at least one pharmaceutically acceptable excipient is a cysteine compound. A cysteine compound described herein encompasses cysteine, cysteine acid salts (e.g. cysteine hydrochloride, or cysteine dihydrochloride), N-substituted cysteines (e.g. N-acetyl cysteine), cysteine esters, cysteine dimers (e.g. cysteine) and cysteine containing peptides. The cysteine may be D or L cysteine or a combination thereof.

In some embodiments, the at least one cysteine compound is present in an amount 0.5-50% w/w of the pharmaceutical composition, or at least 1-35% w/w, or at least 6-30% w/w of the pharmaceutical composition, or at least 10-25% w/w of the pharmaceutical composition. In some embodiments, the at least one cysteine compound is present in an amount greater than 5% w/w of the pharmaceutical composition, or greater than 7.5% w/w, or greater than 10% w/w, or greater than 12% w/w of the pharmaceutical composition. In some embodiments, the at least one cysteine compound is present in an amount less than 30% w/w of the pharmaceutical composition, or less than 25% w/w of the pharmaceutical composition.

In some embodiments, the at least one cysteine compound is selected from cysteine, cysteine hydrochloride, N-acetyl cysteine or glutathione. In some embodiments, the at least one cysteine compound is cysteine hydrochloride and/or N-acetyl cysteine. In some embodiments, the at least one cysteine compound is a combination of cysteine hydrochloride and N-acetyl cysteine.

In some embodiments, the cysteine hydrochloride is present in an amount 0.5-15% w/w of the pharmaceutical composition and/or the N-acetyl cysteine is present in an amount 3-15% w/w of the pharmaceutical composition. In some embodiments, the cysteine hydrochloride is present in an amount 1-13% w/w of the pharmaceutical composition and/or the N-acetyl cysteine is present in an amount 5-13% w/w of the pharmaceutical composition. In some embodiments, the % w/w ratio of cysteine hydrochloride to N-acetyl chloride in the pharmaceutical composition is from 1:1 to about 1:4, or from about 1.75:1 to about 1:1.25. In some embodiments, the amount of cysteine hydrochloride is present in an amount less than 5 wt. %, or less than 4 wt. %, or less than 3 wt. %, or less than 2.5 wt. %, or less than 2 wt. % by weight of the pharmaceutical composition. In some examples, pharmaceutical compositions comprising lower amounts of cysteine hydrochloride (i.e. up to 3% w/w) may improve the stability of the Compound of Formula (I), e.g. Remdesivir and/or the pharmaceutical formulation.

In some embodiments the % w/w ratio of the at least one cysteine compound to the compound of Formula (I) in the pharmaceutical composition is at least 1:1, or optionally greater than 1.25:1, or optionally greater than 1.5:1, or optionally greater than 1.75:1, or optionally greater than 2.1, or optionally greater than 2.25:1, or wherein the % w/w ratio of the at least one cysteine compound to the compound of Formula (I) in the pharmaceutical composition is optionally greater than 2.5:1. In some embodiments, the % w/w at least one cysteine compound to the compound of Formula (I) in the pharmaceutical composition is less than 5:1, or less than 4:1. In some embodiments, the % w/w ratio of the at least one cysteine compound to the compound of Formula (I) in the pharmaceutical composition is from 1:1 to 5:1.

### Acid or Salts Thereof

In some embodiments, the at least one pharmaceutically acceptable excipient is an acid or salt thereof. In some embodiments, the acid is an organic acid selected from lactic acid, adipid acid, acetic acid, citric acid, formic acid, succinic acid, oxalic acid, ascorbic acid, uric acid, malic acid, tartaric acid or any combination thereof. Suitable salts may include, but are not limited to, sodium, potassium, calcium, magnesium, and ammonium. In some embodiments, the at least one acid is present in an amount 1-35% w/w of the pharmaceutical composition, or at least 2-30% w/w, or at least 10-25% w/w, or at least 3-20% w/w, or at least 4-15% w/w of the pharmaceutical composition. In some embodiments, the at least one acid is present in an amount greater than 1% w/w of the pharmaceutical composition, or greater than 2% w/w, or greater than 3% w/w, or greater than 4% w/w, or greater than 5% w/w of the pharmaceutical composition. In some embodiments, the at least one acid is present in an amount less than 30% w/w of the pharmaceutical composition, or less than 25% w/w, or less than 20% w/w, or less than 15% w/w, or less than 10% w/w of the pharmaceutical composition. In some embodiments, the at least one pharmaceutically acceptable excipient may comprise at least one cysteine compound and an acid or salt thereof.

In some embodiments the % w/w ratio of the at least one acid or salt thereof to the compound of Formula (I) in the pharmaceutical composition is at least 1:1, or optionally greater than 1.25:1, or optionally greater than 1.5:1, or optionally greater than 1.75:1, or optionally greater than 2.1, or optionally greater than 2.25:1, or wherein the % w/w ratio of the at least one acid or salt thereof to the compound of Formula (I) in the pharmaceutical composition is optionally greater than 2.5:1. In some embodiments, the % w/w at least one acid or salt thereof to the compound of Formula (I) in the pharmaceutical composition is less than 5:1, or less than 4:1. In some embodiments, the % w/w ratio of the at least one acid or salt thereof to the compound of Formula (I) in the pharmaceutical composition is from 1:1 to 5:1.

### N-Acetyl Amino Acids

In some embodiments, the at least one pharmaceutically acceptable excipient comprises at least one N-acetyl amino acid. In some embodiments, the N-acetyl amino acid is any suitable amino acid. The amino acid may be a natural amino acid or an unnatural amino acid. In some embodiments, the amino acid may be a D or L amino acid or a combination thereof. In some embodiments, the amino acid may be alanine, valine, histidine, methionine, lysine, phenylalanine, threonine, tryptophan, asparagine, aspartic acid, cysteine, glutamine, glutamic acid, proline, serine, leucine, isoleucine, glycine, isoleucine, tyrosine, tryptophan or a combination thereof. In some embodiments, the N-acetyl amino acid is N-acetyl cysteine or N-acetyl alanine. In some embodiments, the at least one pharmaceutically acceptable excipient comprises at least one cysteine compound and at least one N-acetyl amino acid. In some embodiments, the at least one cysteine compound and at the least one N-acetyl amino acid may be the same compound (e.g. N-acetyl cysteine). In other embodiments, the at least one cysteine compound and the at least one N-acetyl amino acid may be different compounds (e.g. N-acetyl cysteine in combination with cysteine hydrochloride, or N-acetyl alanine in combination with cysteine hydrochloride). In some embodiments, the at least one N-acetyl amino acid is present in an amount 0.5-15% w/w of the pharmaceutical composition, or 3-13% w/w of the pharmaceutical composition, or 5-10% w/w of the pharmaceutical composition. In some embodiments, the N-acetyl amino acid is present in an amount greater than 3% w/w, or greater than 4% w/w, or greater than 5% w/w, or greater than 7.5%, or greater than 10% w/w of the pharmaceutical composition. In some embodiments, the at least one pharmaceutical composition comprises the N-acetyl amino acid in an amount less than 15% w/w, or less than 12.5% w/w, or less than 10% w/w, or less than 7.5% w/w of the pharmaceutical composition. The N-acetyl amino acid may be used as an acidulant.

### Amino Acid Hydrochlorides

In some embodiments, the at least one pharmaceutically acceptable excipient is an amino acid hydrochloride (i.e. an amino acid hydrochloride salt). The amino acid may be a natural amino acid or an unnatural amino acid. In the amino acid may be a D or L amino acid or a combination thereof. In some embodiments, the amino acid hydrochloride is selected from cysteine hydrochloride, glycine hydrochloride or glutamic acid hydrochloride. In some embodiments, the amino acid hydrochloride is cysteine hydrochloride.

In some embodiments, the amino acid hydrochloride is present in an amount 0.5-15% w/w of the pharmaceutical composition. In some embodiments, the amino acid hydrochloride is present in an amount greater than or greater than 1% w/w, 2% w/w, or greater than 3% w/w, or greater than 4% w/w, or greater than 5% w/w, or greater than 7.5%, or greater than 10% w/w of the pharmaceutical composition. In some embodiments, the at least one pharmaceutical composition comprises the amino acid hydrochloride is present in an amount less than 15% w/w, or less than 12.5% w/w, or less than 10% w/w, or less than 7.5% w/w of the pharmaceutical composition. The amino acid hydrochloride may be used as an acidulant.

In some embodiments, the at least one pharmaceutically acceptable excipient comprises at least one cysteine compound and at least one amino acid hydrochloride. In some embodiments, the at least one cysteine compound and the least one amino acid hydrochloride may be the same compound (e.g. cysteine hydrochloride). In other embodiments, the at least one cysteine compound and the at least one amino acid may be different compounds (e.g. glycine hydrochloride and N-acetyl cysteine).

### Absence of Cyclodextrin

In some embodiments, the pharmaceutical composition is free from cyclodextrins. In some embodiments, the pharmaceutical composition is free from alpha, beta, and/or gamma cyclodextrins. In some embodiments, the pharmaceutical composition is free from any derivatized and/or modified cyclodextrins, such as, hydroxylpropyl β-cyclodextrin and sulfobutylether β-cyclodextrin.

### Liquid Formulation and Co-Solvents

In some embodiments, the pharmaceutical composition is a liquid formulation. In some embodiments, the liquid formulation has a pH of less than 8.5, optionally less than 7.9, or optionally less than 7.8, or optionally less than 7.7, or optionally less than 7.6, or optionally less than 6.5, or optionally less than 6.0, or optionally less than 5.5. In some embodiments, the liquid formulation has a pH of more than 1, optionally more than 1.2, or optionally more than 1.4, or optionally more than 1.6, or optionally more than 1.8, or optionally more than 2.0, or optionally more than 2.5, or optionally more than 3.0, or optionally more than 3.5, or optionally more than 4.0. In some embodiments, the optionally wherein the pH is in the range of 1-<8.

In some embodiments, pharmaceutical composition comprises one or more co-solvents. The co-solvent may be termed a solubilizing agent. In some embodiments, the pharmaceutical composition comprises one, two, three, or four or more co-solvents. The co-solvent may be selected from polyethylene glycol (PEG) (e.g. PEG 400, PEG 300, PEG 600), glycerol, DMSO, ethanol, propylene glycol, polypropylene glycol, N-methylpyrrolidone, benzyl alcohol, cetostearyl alcohol, benzylbenzoate, corn syrup, acacia syrup, glucose syrup, acetyltributyl citrate, lactic acid, acetic acid, ethylacetate, benzoic acid, polyoxyl 35 castor oil, polysorbate 20, 40, and 80; water, peppermint oil, or a combination thereof. In some embodiments, the one or more co-solvents is or comprises a polar solvent. In some embodiments, the polar solvents may be an aprotic solvent or a protic solvent. In some embodiments, the one or more co-solvents may be free from oils. In some embodiments, the co-solvent may be selected from polyethylene glycol (PEG) (e.g. PEG 300, PEG 400), N-methyl pyrrolidone, propylene glycol, water benzyl alcohol, ethanol, povidone, peppermint oil or a polyvinyl caprolactam-polyvinyl acetate-polyethylene glycol graft co-polymer, or a combination thereof. In some embodiments, the one or more solvents comprises a polymeric solvent.

In some embodiments, pharmaceutical composition comprises one or more solubility enhancing solid polymers, as a polyvinyl caprolactam-polyvinyl acetate-polyethylene glycol graft co-polymer (Soluplus) or high molecular weight polyethylene glycols (MW 600), or a block copolymer of poly(ethylene oxide) (PEO) and poly(propylene oxide) (PPO), such as, poloxamer.

In some examples, the pharmaceutical composition comprises at least 0.1 wt. % co-solvent by weight, or at least 0.5 wt. %, or at least 1 wt. %, or at least 5 wt. %, or at least 10 wt. %, or at least 20 wt. %, or at least 30 wt. %, or at least 40 wt. % co-solvent by weight of the total pharmaceutical composition, or at least 50% co-solvent by weight of the total pharmaceutical composition, or at least 60% co-solvent by weight of the total pharmaceutical composition, or at least 50% co-solvent by weight of the total pharmaceutical composition, or at least 60% co-solvent by weight of the total composition. In some embodiments, the pharmaceutical composition comprises less than 80% co-solvent by weight of the total pharmaceutical composition, or less than 75%, or less than 70%, or less than 65%. In some embodiments, the pharmaceutical composition comprises 50-86% w/w of one or more co-solvent, or 55-86% w/w of one or more co-solvent, or 62-86% w/w of one or more co-solvent.

In some embodiments, the one or more solvents comprises a polymeric solvent. In some embodiments, the polymeric solvent has a molecular weight greater than 200, or greater than 225, or greater than 250, or a molecular weight greater than 275. In some embodiments, the one or more co-solvents comprises PEG, optionally wherein the PEG is present in an amount >40% w/w of the pharmaceutical composition, or >50% w/w of the pharmaceutical composition, or >50% w/w of the pharmaceutical composition. PEG is present in an amount >60% w/w of the formulation. In some embodiments, the one or more co-solvents comprises PEG in an amount from 40-90% by w/w of the pharmaceutical composition. In some embodiments, the PEG has a molecular weight from 200 to 600.

In some embodiments, the one or more co-solvent comprises PEG 300 and/or PEG 400. The number following the PEG is indicative of the average molecular weight of the polymer. In some embodiments, the one or more co-solvent comprises PEG 300 and PEG 400. In some embodiments, the pharmaceutical composition comprises 10-30% w/w of PEG 300 and 35-65% w/w PEG 400. In some embodiments, the ratio of PEG 400:PEG 300 is greater than 1:1, or greater than 1.5:1, or greater than 2:1.

In some embodiments, the one or more co-solvents is selected from PEG, benzyl alcohol, ethanol, or a combination thereof.

In some embodiments, the one or more solvent comprises one or more alcohol compound, e.g., a C2-C7 alcohol compound. In some embodiments, the one or more alcohol compound may be selected from ethanol, benzyl alcohol, glycerol, tertiary butyl alcohol (or tert-butyl alcohol) or polyethylene glycol or propylene glycol.

In some embodiments, the one or more co-solvent comprises one or more PEG compounds (e.g. PEG 300 and PEG 400) in combination with one or more alcohol compounds. In some embodiments, the one or more alcohol compounds comprise benzyl alcohol and/or ethanol. In some embodiments, the one or more co-solvents comprises PEG in an amount from 40-90% w/w of the pharmaceutical composition and one or more alcohol compounds in an amount from 2-12% w/w of the pharmaceutical composition. In some embodiments, the pharmaceutical composition comprises 49-90% w/w PEG, 2-8% w/w ethanol and/or 2-8% w/w benzyl alcohol. In some embodiments, the pharmaceutical composition comprises 49-90% w/w PEG, less than 4.5% w/w ethanol and/or 2-8% w/w benzyl alcohol. In some embodiments, the pharmaceutical composition comprises 49-90% w/w PEG, and 2-8% w/w benzyl alcohol. In some embodiments, the pharmaceutical composition may comprise less than 4.5% w/w, or less than 3% w/w ethanol, or less than 2% w/w ethanol, or less than 1% w/w ethanol, or be free of ethanol.

In some embodiments, the concentration of the compound of Formula (I) in the pharmaceutical composition, (i.e. wherein the pharmaceutical composition is as a liquid formulation), is from 30 mg/mL to 100 mg/mL, or from 40 mg/mL to 80 mg/mL, or from 50 mg/mL to 75 mg/mL.

### Surfactant

In some embodiments, the pharmaceutical composition comprises a surfactant. In some embodiments, the pharmaceutical composition comprises one, two, three, or four or more surfactants. In some examples, the pharmaceutical composition comprises at least 0.1 wt. % surfactant(s) by weight, or at least 0.5 wt. %, or at least 1 wt. %, or at least 5 wt. %, or at least 10 wt. %, or at least 20 wt. % surfactant(s) by weight of the total pharmaceutical composition. In some embodiments, the pharmaceutical composition comprises less than 20 wt. % surfactant(s) by weight of the total pharmaceutical composition, or less than 10 wt. %, or less than 8. wt. %, or less than 7 wt. % surfactant(s) by weight of the total pharmaceutical composition. In some embodiments, the pharmaceutical composition comprises 1-20% w/w surfactant, or 2-15% w/w surfactant, or 3-10% w/w surfactant, or 4-8% w/w surfactant, or 5-7% w/w surfactant, or about 6 w/w % surfactant.

In some embodiments, the one or more surfactant(s) is selected from polysorbate 20, polysorbate 40, polysorbate 60, polysorbate 80, polyoxyl 35 castor oil, cremophor, polyoxyethylene (20) sorbitan monooleate, polyethylene glycol sorbitan monooleate, polyoxyethylenesorbitan monooleate, or a block copolymer of poly(ethylene oxide) (PEO) and poly(propylene oxide) (PPO), such as, poloxamer, or a combination thereof.

In some embodiments, the one or more surfactant(s) are non-ionic surfactants. In some embodiments, the one or more surfactant(s) have a weight average molecular weight of less than 5000, or less than 3000, or less than 1500. In some embodiments, the one or more surfactant(s) have a weight average molecular weight from 1000 to 1500. In some embodiments, the one or more surfactant(s) is selected from a polysorbate 80 and poloxamer. In some embodiments, the one or more surfactants is or comprises a polysorbate, for example, polysorbate 20 (polyoxyethylene (20) sorbitan monolaurate) polysorbate 40 (polyoxyethylene (20) sorbitan monopalmitate), polysorbate 60 (polyoxyethylene (20) sorbitan monostearate) or polysorbate 80 (polyoxyethylene (20) sorbitan monooleate). In an embodiment, the polysorbate is polysorbate 80. Polysorbate 80 is otherwise known and distributed as Tween 80. In some embodiments, the one or more surfactants is or comprises a poloxamer, e.g., poloxamer 188. In some embodiments, the pharmaceutical composition may comprise two surfactants. In some examples, one surfactant is used to aid solubility of the Compound of Formula (I) and the other surfactant may be used to stabilize the Compound of Formula (I). In some embodiments, the pharmaceutical composition may comprise a polysorbate (e.g. polysorbate 80) in combination with a triblock copolymer, (e.g. poloxamer such as poloxamer 188).

In some embodiments, the HLB value of the one or more surfactant (i.e. at least one of the one or more surfactants) is from 10-20, optionally from 12 to 18, or optionally from 14 to 17, or optionally from 14.5 to 15.5. In some embodiments, the HLB value of the one or more surfactant is greater than 10, or greater than 12, or greater than 14. In some embodiments, the HLB value of the one or more surfactant is less than 20, or less than 18, or less than 17, or less than 16 (e.g. polysorbate 80).

In some examples, the pharmaceutical composition comprises at least 0.1 wt. % polysorbate(s) (polysorbate 80) by weight, or at least 0.5 wt. %, or at least 1 wt. %, or at least 5 wt. %, or at least 10 wt. %, or at least 20 wt. % polysorbate(s) (e.g. polysorbate 80) by weight of the total pharmaceutical composition. In some embodiments, the pharmaceutical composition comprises less than 20 wt. % polysorbate(s) (e.g. polysorbate 80) by weight of the total pharmaceutical composition, or less than 10 wt. %, or less than 8. wt. %, or less than 7 wt. % polysorbate(s) by weight of the total pharmaceutical composition. In some embodiments, the pharmaceutical composition comprises 1-20% w/w polysorbate, or 2-15% w/w polysorbate, or 3-10% w/w polysorbate, or 4-8% w/w polysorbate, or 5-7% w/w polysorbate, or about 6 w/w % polysorbate by weight of the pharmaceutical composition (e.g. polysorbate 80). In some examples, the pharmaceutical composition comprises at least 0.1 wt. % poloxamer(s) (poloxamer 188) by weight, or at least 0.5 wt. %, or at least 1 wt. %, or at least 5 wt. %, poloxamer(s) (e.g. poloxamer 188) by weight of the total pharmaceutical composition. In some embodiments, the pharmaceutical composition comprises less than 20 wt. % poloxamer(s) by weight of the total pharmaceutical composition, or less than 10 wt. %, or less than 8. wt. %, or less than 7 wt. %, or less than 5 wt. %, or less than 4 wt. %, or less than 3 wt. %, or less than 2.5 wt. %, or less than 2 wt. % poloxamer (s) by weight of the total pharmaceutical composition. In some embodiments, the pharmaceutical composition comprises from about 0.25 wt. % to about 5 wt. % poloxamer by weight of the total pharmaceutical composition, or from about 0.5 wt. % to about 4 wt. % poloxamer, or from 1 wt. % to about 3 wt. % poloxamer (e.g. poloxamer 188) by weight of the total pharmaceutical composition. In some embodiments, these stated amounts of polysorbate and poloxamer can be readily combined.

### Compound of Formula (I)

The pharmaceutical composition comprises a compound of Formula (I)

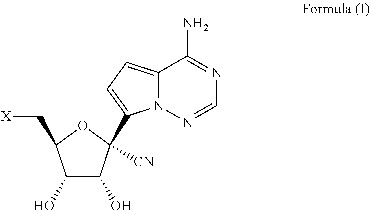

or a pharmaceutically acceptable salt thereof;
wherein X is selected from a hydroxyl, a metal salt hydroxylate, an O-linked phosphoester, an 0-linked phosphoramidite, an O-linked ester or an O-linked carbamate, an S-linked phosphothioate, or an N-linked phosphoramidite.

In some embodiments, the compound of Formula (I) is a prodrug.

In some embodiments, X is an O-linked phosphoramidite.

In some embodiments, X is

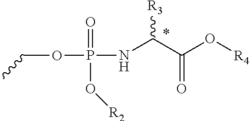

wherein R₂ is H, lithium, sodium, potassium, aluminium, ammonium, arginine benzathine, calcium, chloroprocaine, choline, diethanolamine, ethanolamine, ethylenediamine, lysine, magnesium, histidine, tromethamine, meglumine, procaine, trimethylamine, zinc, C₁-C₆ alkyl, C₂-C₆-allyl, C₂-C₆ alkenyl, phenyl, biphenyl, heteroaryl,
R₃ is H or C₁-C₆ alkyl; and
R₄ is H, lithium, sodium, potassium, aluminium, ammonium, arginine benzathine, calcium, chloroprocaine, choline, diethanolamine, ethanolamine, ethylenediamine, lysine, magnesium, histidine, tromethamine, meglumine, procaine, trimethylamine, zinc, C₁-C₆ alkyl, C₂-C₆ allyl, or C₂-C₆ alkenyl, phenyl, biphenyl, heteroaryl.

In some examples, R₃ may be selected from H, methyl, ethyl, isopropyl, isobutyl, or secbutyl. In some examples, R₃ is methyl.

In some embodiments, R₂ is phenyl,

R₃ is H or C₁-C₆ alkyl, e.g., methyl, and
R₄ is H, lithium, sodium, potassium, aluminium, ammonium, arginine benzathine, calcium, chloroprocaine, choline, diethanolamine, ethanolamine, ethylenediamine, lysine, magnesium, histidine, tromethamine, meglumine, procaine, trimethylamine, zinc, C₁-C₆ alkyl, C₂-C₆ allyl, or C₂-C₆ alkenyl, phenyl, biphenyl, heteroaryl, in some examples, wherein C₁-C₆ alkyl is selected from methyl, ethyl, n-propyl, i-propyl, n-butyl, s-butyl, i-butyl, t-butyl, n-pentyl, i-pentyl, s-pentyl pentyl, neopentyl, 3-pentyl, secisopentyl or 2-methylbutyl.

In some embodiments, R₂ is H, lithium, sodium, potassium, aluminium, ammonium, arginine benzathine, calcium, chloroprocaine, choline, diethanolamine, ethanolamine, ethylenediamine, lysine, magnesium, histidine, tromethamine, meglumine, procaine, trimethylamine, zinc, C₁-C₆ alkyl, C₂-C₆-allyl, C₂-C₆ alkenyl, phenyl, biphenyl, heteroaryl

R₃ is H or C₁-C₆ alkyl, e.g., methyl, and
R₄ is methyl, ethyl, n-propyl, i-propyl, n-butyl, s-butyl, i-butyl, t-butyl, h-pentyl, i-pentyl, s-pentyl, t-pentyl, neopentyl, 3-pentyl, secisopentyl or 2-methylbutyl, for example, 3-pentyl.

In some embodiments, the chiral center (*) has R or S stereochemistry. In other words, X=

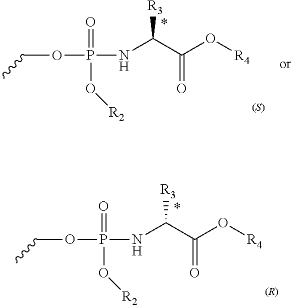

In some examples, X has R stereochemistry. In these examples, the stereochemistry is different to that of Remdesivir, which in some examples leads to improved bioavailability.

In some embodiments, X=

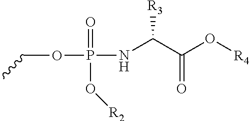

wherein R₂ is H, lithium, sodium, potassium, aluminium, ammonium, arginine benzathine, calcium, chloroprocaine, choline, diethanolamine, ethanolamine, ethylenediamine, lysine, magnesium, histidine, tromethamine, meglumine, procaine, trimethylamine, zinc, C₁-C₆ alkyl, C₂-C₆ allyl, or C₂-C₆ alkenyl, phenyl, biphenyl, heteroaryl,
R₃ is H, methyl, ethyl, isopropyl, isobutyl, secbutyl, phenyl,
R₄ is H, lithium, sodium, potassium, aluminium, ammonium, arginine benzathine, calcium, chloroprocaine, choline, diethanolamine, ethanolamine, ethylenediamine, lysine, magnesium, histidine, tromethamine, meglumine, procaine, trimethylamine, zinc, C₁-C₆ alkyl, C₂-C₆ allyl, or C₂-C₆ alkenyl, phenyl, biphenyl, heteroaryl.

In some embodiments, X=

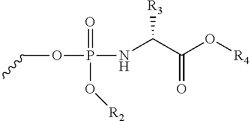

wherein R₂ is H, Li, Na, K, C₁-C₆ alkyl, C₂-C₆-allyl, C₂-C₆ alkenyl, phenyl, biphenyl, heteroaryl,
R₃ is H, methyl, ethyl, isopropyl, isobutyl, secbutyl, phenyl,
R₄ is H, Na, Li, K, C₁-C₆ alkyl, C₂-C₆ allyl, or C₂-C₆ alkenyl, phenyl, biphenyl, heteroaryl.

In all of the above examples, R₂, R₃ and R₄ are selected such that the log P of the compound of Formula (I) is less than 2, or less than 1.8, or less than 1.6, thereby having a lower log P and lower lipophilicity than Remdesivir. In some examples, a compound of Formula (I) with a lower lipophilicity than Remdesivir was found to have a higher solubility than Remdesivir. In some examples, R₂, R₃ and R₄ are selected such that the log S is greater than −3, or −2.5, or −2, or −1.5. For example, R₂ may be selected from H, methyl, ethyl or heteroaryl. In some examples, R₃ may be H. In some examples, R₄ may be selected from H, methyl, ethyl, or heteroaryl.

In some embodiments, X=

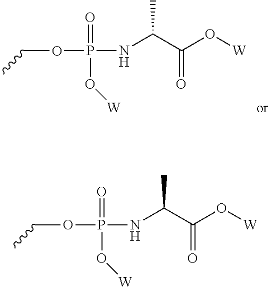

wherein W may be selected from H, lithium, sodium, potassium, aluminium, ammonium, arginine benzathine, calcium, chloroprocaine, choline, diethanolamine, ethanolamine, ethylenediamine, lysine, magnesium, histidine, tromethamine, meglumine, procaine, trimethylamine, zinc, a combination thereof.

In some embodiments, the O-linked phosphoramidite can be made by following a similar synthesis to that which is known for Remdesivir, e.g., as described in J. Med. Chem. 2017, 60, 5, 1648-166, the contents of which are incorporated by reference, wherein the R₂, R₃ and R₄ groups of building blocks are altered as appropriate.

In some embodiments, the O-linked phosphoramidite has the following structure (i.e. Remdesivir, Compound (A), otherwise known as GS 5734). In some examples, the Compound of Formula (I) is Remdesivir.

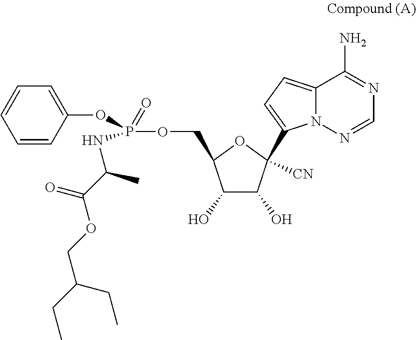 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

In some embodiments, the O-linked phosphoramidite has the following structure, Compound (B), otherwise known as GS-6620.

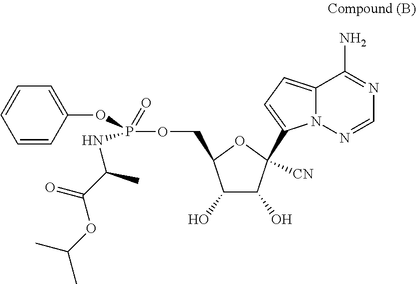 [SMILES: CC(C)OC(=O)[C@H](C)N[P@](=O)(OC[C@H]1O[C@@](C#N)(c2ccc3c(N)ncnn23)[C@H](O)[C@@H]1O)Oc1ccccc1]

In some embodiments, X is an O-linked phosphoester or an O-linked phosphoramidite with the following structure—Formula (II).

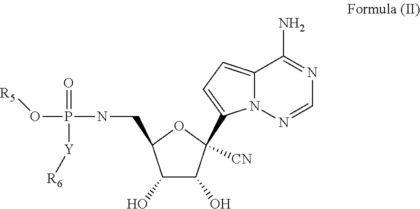

wherein Y is O or NH, and wherein R₅ and R₆ may each be independently selected from C₁-C₆ alkyl, C₂-C₆ allyl, or C₂-C₆ alkenyl, phenyl, biphenyl, heteroaryl.

In some embodiments, X is a hydroxyl group and the compound of Formula (I) has the structure as shown compound (C). Compound (C) can be made following the synthesis as described in J. Med. Chem. 2017, 60, 5, 1648-166, the contents of which are incorporated by reference. This compound is otherwise known as GS-441524.

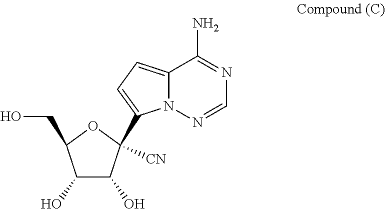 [SMILES: N#C[C@@]1(c2ccc3c(N)ncnn23)O[C@H](CO)[C@@H](O)[C@H]1O]

In some embodiments of the compound of Formula (I), X is a salt hydroxylate. In some embodiments, the salt is selected from lithium, sodium, potassium, aluminium, ammonium, arginine benzathine, calcium, chloroprocaine, choline, diethanolamine, ethanolamine, ethylenediamine, lysine, magnesium, histidine, tromethamine, meglumine, procaine, trimethylamine, zinc a combination thereof. In some embodiments X is a metal salt hydroxylate, for example, wherein the metal salt is Li, Na, Ca, Mg, Zn, or K.

In some embodiments, X is an O-linked ester. In some embodiments of X being an O-linked ester, X is an O-linked amino acid or an O-linked peptide. The O-linked peptide may comprise any number of amino adds, for example, from 2-10 amino adds. In some embodiments, the O-linked peptide is a dipeptide (i.e. 2 amino acids), a tripeptide (i.e. 3 amino acids), or a tetrapeptide (i.e. 4 amino acids). In some embodiments, the O-linked peptide or O-linked amino acid is formed from any suitable natural or unnatural amino acid. In some embodiments, the amino acid is an L-amino acid, D-amino acid, or a combination thereof. In some embodiments, the amino acid is selected from alanine, valine, histidine, methionine, lysine, phenylalanine, threonine, tryptophan, asparagine, aspartic acid, cysteine, glutamine, glutamic acid, proline, serine, leucine, isoleucine, glycine, isoleucine, tyrosine, tryptophan or a combination thereof. In some embodiments, the amino acid is selected from alanine, valine, leucine, isoleucine, glycine, isoleucine, tyrosine, tryptophan or a combination thereof. In some embodiments, the amino acids selected from alanine, valine, leucine, isoleucine, glycine, isoleucine, tyrosine, tryptophan or a combination thereof.

In some embodiments X is an O-linked ester and the compound is of Formula (III)

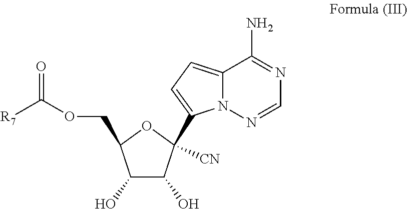

wherein R₇ may be selected from C₁-C₆ alkyl, C₂-C₆ allyl, or C₂-C₆ alkenyl, phenyl, biphenyl, heteroaryl. The compound of Formula (I) comprising X as an O-linked ester may be formed by selectivity esterifying the compound of Formula (I) wherein X is a hydroxyl group. In some examples, the compound of Formula (I) wherein X is a hydroxyl group may be reacted with the appropriate acid chloride (e.g. R₇—C(═O)—Cl) or acid anhydride (R₇—O—C(═O)—O—C(C═O)—R₇) or any other suitable esterification method known in the art.

In some embodiments, X is an O-linked carbamate in some embodiments X is an O-linked carbamate and the compound is of Formula (IV):

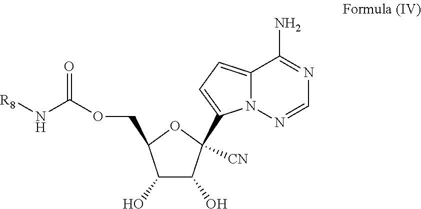

wherein R₈ may be selected from H, C₁-C₆ alkyl, C₂-C₆ allyl, or C₂-C₆ alkenyl, phenyl, biphenyl or heteroaryl.

In some embodiments, X is an S-linked phosphothioate. In some embodiments X is an S-linked phosphothioate and the compound is of Formula (V):

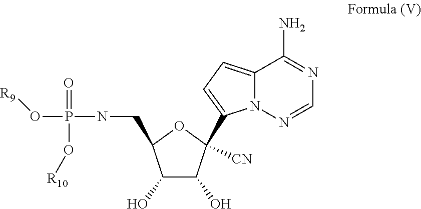

wherein R₉ and R₁₀ may each be selected from H, C₁-C₆ alkyl, C₂-C₆ allyl, or C₂-C₆ alkenyl, phenyl, biphenyl or heteroaryl.

In some embodiments, X is an S-linked phosphothioate, X may be:

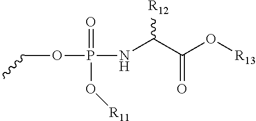

wherein R₁₁ is H, lithium, sodium, potassium, aluminium, ammonium, arginine benzathine, calcium, chloroprocaine, choline, diethanolamine, ethanolamine, ethylenediamine, lysine, magnesium, histidine, tromethamine, meglumine, procaine, trimethylamine, zinc, C₁-C₆ alkyl, C₂-C₆-allyl, C₂-C₆ alkenyl, phenyl, biphenyl, heteroaryl,
R₁₂ is H, methyl, ethyl, isopropyl, isobutyl, secbutyl, phenyl,
R₁₃ is H, lithium, sodium, potassium, aluminium, ammonium, arginine benzathine, calcium, chloroprocaine, choline, diethanolamine, ethanolamine, ethylenediamine, lysine, magnesium, histidine, tromethamine, meglumine, procaine, trimethylamine, zinc, C₁-C₆ alkyl, C₂-C₆ allyl, or C₂-C₆ alkenyl, phenyl, biphenyl, heteroaryl.

In some embodiments, X is an N-linked phosphoramidite. In some embodiments X is an N-linked phosphoramidite and the compound is of Formula (VI):

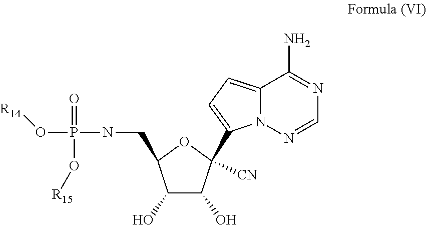

wherein R₁₄ and R₁₅ may each be selected from H, C₁-C₆ alkyl, C₂-C₆ allyl, or C₂-C₆ alkenyl, phenyl, biphenyl, heteroaryl.

In some embodiments, X is an N-linked phosphoramidite, X may be:

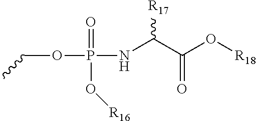

wherein R₁₆ is H, lithium, sodium, potassium, aluminium, ammonium, arginine benzathine, calcium, chloroprocaine, choline, diethanolamine, ethanolamine, ethylenediamine, lysine, magnesium, histidine, tromethamine, meglumine, procaine, trimethylamine, zinc, C₁-C₆ alkyl, C₂-C₆ allyl, or C₂-C₆ alkenyl, phenyl, biphenyl, heteroaryl,
R₁₇ is H, methyl, ethyl, isopropyl, isobutyl, secbutyl, phenyl,
R₁₈ is H, lithium, sodium, potassium, aluminium, ammonium, arginine benzathine, calcium, chloroprocaine, choline, diethanolamine, ethanolamine, ethylenediamine, lysine, magnesium, histidine, tromethamine, meglumine, procaine, trimethylamine, zinc, C₁-C₆ alkyl, C₂-C₆ allyl, or C₂-C₆ alkenyl, phenyl, biphenyl, heteroaryl.

In some embodiments, for example, wherein X is a hydroxyl, a metal salt hydroxylate, an O-linked phosphoester, an O-linked phosphoramidite, an O-linked ester or an O-linked carbamate, the metabolized active product of the compound of Formula (I) is Compound (D)

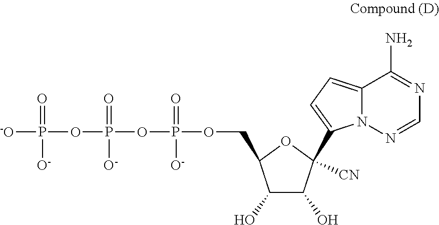

In other words, the compound of Formula (I) is capable of being metabolized to the compound (D) in the human or animal cell. Compound (D) is otherwise known as GS-443902. The compound of Compound (D) may be in ionized or non-ionized form.

In some embodiments, the compound is capable of being metabolized to the monophosphate Compound (E) in the human or animal cell. Compound (E) is capable of being phosphorylated twice in the human or animal cell by one or more kinase enzymes. Compounds of Formula (I) wherein X is O-linked phosphoramidite and O-linked phosphoester can be first metabolised to Compound (E) by hydrolytic degradation, and thereby do not require monophosphorylation in the human or animal cell, a step that could be potentially rate-limiting.

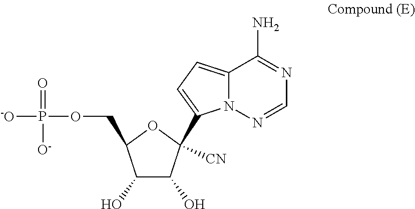

In some embodiments, for example, wherein X is:

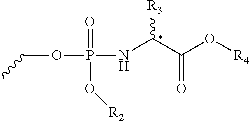

the compound of Formula (I) is metabolised to a compound of Formula (VII)

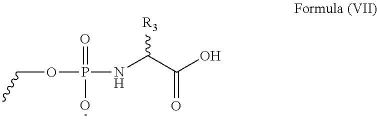

In some embodiments for example, wherein X is OH, a hydroxylate salt, an O-linked phosphoester or an O-linked phosphoramidite, an O-linked ester, an O-linked carbamate, the compound of Formula (I) is capable of being metabolised to the free hydroxyl (e.g. Compound (C)). In some embodiments, the compound of Formula (I) is metabolised to the free hydroxyl by esterase or amidase enzymes. In some embodiments, the compound of Formula (I) is metabolised to the free hydroxyl by hydrolytic degradation. The free hydroxyl is capable of being phosphorylated three times in the human or animal cell by one or more kinase enzymes, first to that of the monophosphate (i.e. Compound (E)), which is then further phosphorylated to the triphosphate (i.e. Compound (D)).

In some embodiments, wherein X is an S-linked phosphothiaote, the metabolised active product of the compound of Formula (I) is Compound (F):

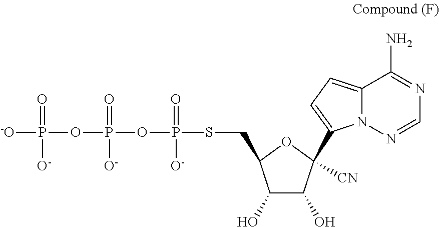

In some embodiments, wherein X is an N-linked phosphoramidite, the metabolized active product of the compound of Formula (I) is Compound (G)

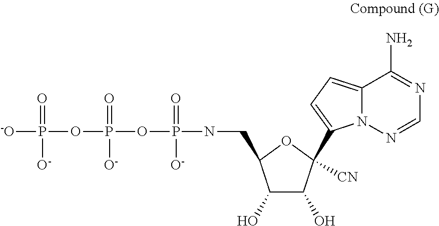

In some embodiments, the compound of Formula (I) has any suitable log P. In some embodiments, the compound of Formula (I) has a log P of less than 2.2. Remdesivir has a log P of 2.1. In alternative embodiments, the compound of Formula (I) has a log P of less than 2, or less than 1.8, or less than 1.6, or less than 1.4, or less than 1.2, or less than 1. In some embodiments, the compound of Formula (I) has a log P of greater than −1, or greater than −0.5, or greater than 0. In some embodiments, the compound of Formula (I) has a log P between −1 and 2.2, or between −1 and 2, or from 0 to 2.2, or between to 0 to 2.

In some embodiments, the pharmaceutical composition comprises up to 15% w/w of Compound of Formula (I), or up to 14%, or up to 13%, or up to 12%, or up to 11%, or up to 10%, or up to 9% w/w, or up to 8% w/w, or up to 7% w/w of compound of Formula (I). In some embodiments, the pharmaceutical composition comprises 0.05-20% w/w of compound of Formula (I), or 1-18% w/w, or 2-16% w/w, or 3-14% w/w, or 4-12% w/w, or 5-10% w/w, or 6-8% w/w of compound of Formula (I).

In example pharmaceutical compositions, the compound of Formula (I) is Remdesivir.

Example Pharmaceutical Compositions

In some embodiments, the pharmaceutical composition may comprise 0.05-20% w/w compound of Formula (I), optionally 4-10% w/w, or optionally 5-7% w/w of compound of Formula (I), 0.5-50% w/w of at least one cysteine compound

In some embodiments, the pharmaceutical composition may comprise 3-20% w/w compound of Formula (I), optionally 4-10% w/w, or optionally 5-7% w/w of compound of Formula (I), 1-35% w/w of at least one cysteine compound

In some embodiments, the pharmaceutical composition may comprise 3-20% w/w compound of Formula (I), optionally 4-8% w/w, or optionally 5-7% w/w of compound of Formula (I), 1-30% w/w of at least one cysteine compound

In some embodiments, the pharmaceutical composition may comprise 3-20% w/w compound of Formula (I), optionally 4-8% w/w, or optionally 5-7% w/w of compound of Formula (I), 1-50% w/w of at least one cysteine compound

In some embodiments, the pharmaceutical composition may comprise 3-10% w/w of compound of Formula (I), optionally 4-8% w/w, or optionally 5-7% w/w of compound of Formula (I), 0.5-35% at least one cysteine compound, 50-86% w/w of one or more co-solvent

In some embodiments, the pharmaceutical composition may comprise 3-10% w/w of compound of Formula (I), optionally 4-8% w/w, or optionally 5-7% w/w of compound of Formula (I), 1-30% cysteine compound, 50-86% w/w of one or more co-solvent

In some embodiments, the pharmaceutical composition may comprise 3-10% w/w of compound of Formula (I), optionally 4-8% w/w, or optionally 5-7% w/w of compound of Formula (I),

1-30% cysteine compound,
50-86% w/w of one or more co-solvent
2-8% w/w surfactant

In some embodiments, the pharmaceutical composition may comprise

3-10% w/w of compound of Formula (I), optionally 4-8% w/w, or optionally 5-7% w/w of compound of Formula (I),
1-30% cysteine compound,
50-86% w/w of one or more co-solvent
2-8% w/w polysorbate 80

In some embodiments, the pharmaceutical composition may comprise

3-10% w/w of compound of Formula (I), optionally 4-8% w/w, or optionally 5-7% w/w of compound of Formula (I),
0.5-15% w/w cysteine hydrochloride monohydrate,
0.5-15% w/w N-acetyl cysteine

In some embodiments, the pharmaceutical composition may comprise

3-10% w/w of compound of Formula (I), optionally 4-8% w/w, or optionally 5-7% w/w of compound of Formula (I),
1-15% w/w cysteine hydrochloride monohydrate,
1-15% w/w N-acetyl cysteine
50-86% w/w one or more co-solvent

In some embodiments, the pharmaceutical composition may comprise

3-10% w/w of compound of Formula (I), optionally 4-8% w/w, optionally 5-7% w/w of compound of Formula (I)
1-15% w/w cysteine hydrochloride monohydrate,
1-15% w/w N-acetyl cysteine
50-83% w/w one or more co-solvent
2-8% w/w surfactant

In some embodiments, the pharmaceutical composition may comprise

3-10% w/w of compound of Formula (I), optionally 4-8% w/w, optionally 5-7% w/w of compound of Formula (I)
1-15% w/w cysteine hydrochloride monohydrate,
1-15% w/w N-acetyl cysteine
50-83% w/w one or more co-solvent
2-8% w/w polysorbate 80

In some embodiments, the pharmaceutical composition may comprise

3-10% w/w of compound of Formula (I), optionally 4-8% w/w of compound of Formula (I), or 5-7% w/w of compound of Formula (I).
1-15% w/w cysteine hydrochloride monohydrate,
1-15% w/w N-acetyl cysteine
2-8% w/w polysorbate 80,

### 35-65% w/w PEG 400

### 10-30% w/w PEG 300

2-12% C2 to C7 alcohol

In some embodiments, the pharmaceutical composition may comprise

3-10% w/w of compound of Formula (I), optionally 4-8% w/w of compound of Formula (I), or 5-7% w/w of compound of Formula (I).
1-15% w/w cysteine hydrochloride monohydrate,
2-8 w/w N-acetyl cysteine
2-8% w/w polysorbate 80,

### 35-65% w/w PEG 400

### 10-30% w/w PEG 300

2-6% w/w ethanol
2-6% w/w benzyl alcohol

In some embodiments of the above Example formulations, the pharmaceutical composition further comprises a poloxamer, e.g. poloxamer 188. The pharmaceutical composition may comprise 0.5-5% w/w poloxamer, e.g. poloxamer 188. The presence of a poloxamer may increase the stability of the Compound of Formula (I), e.g., Remdesivir.

For example, the pharmaceutical composition may comprise

3-10% w/w of compound of Formula (I), optionally 4-8% w/w, or optionally 5-7% w/w of compound of Formula (I),
1-30% cysteine compound,
50-86% w/w of one or more co-solvent
2-8% w/w polysorbate 80
0.5-5% w/w poloxamer 188

For example, the pharmaceutical composition may comprise

3-10% w/w of compound of Formula (I), optionally 4-8% w/w, optionally 5-7% w/w of compound of Formula (I)
1-15% w/w cysteine hydrochloride monohydrate,
1-15% w/w N-acetyl cysteine
50-83% w/w one or more co-solvent
2-8% w/w polysorbate 80
0.5-5% w/w poloxamer 188

In some embodiments of the above Example formulations, the pharmaceutical composition may further comprise a buffer such as tris (i.e. tromethamine). The pharmaceutical composition may comprise 0.25-5% w/w tris.

For example, the pharmaceutical composition may comprise

In some embodiments, the pharmaceutical composition may comprise

3-10% w/w of compound of Formula (I), optionally 4-8% w/w, or optionally 5-7% w/w of compound of Formula (I),
1-30% cysteine compound,
50-86% w/w of one or more co-solvent
2-8% w/w surfactant
0.25-5% w/w tris

In some examples of the above Example formulations, the compound of Formula (I) is Remdesivir.

In some embodiments the pharmaceutical composition may comprise one or more anti-oxidants.

In some embodiments, the pharmaceutical composition is formulated such that the compound of Formula (I) has a solubility of greater than 0.01 mg/mL when placed in an aqueous solution at a pH 6.5, optionally greater than 0.05 mg/mL, optionally greater than 0.1 mg/mL when placed in an aqueous solution at a pH of 6.5

### Formulation

The pharmaceutical composition described herein may be formulated as any suitable formulation for therapeutic use.

In some embodiments, the pharmaceutical composition is an oral formulation. The oral formulation is in the form of a solid oral dosage form, a liquid oral dosage form, a capsule, a tablet, a liquid-filled capsule, a caplet, a chewable gum, an oral film, an oral solution, a suspension, an emulsion, a lozenge, a wafer, a granulated powder formulation, a simple powder or mixture thereof, an elixir or a syrup that is capable of delivering the exact dose consistently to achieve adequate plasma concentrations of the compound of Formula (I) to bring about the intended therapeutic effect. In some embodiments, the tablet is an immediate release formulation. In some embodiments the tablet is a film-coated tablet. In some embodiments the tablet is an orally disintegrating tablet (ODT).

In some embodiments, the pharmaceutical composition is a liquid formulation. In some embodiments, the liquid formulation is used in an injectable solution. In some embodiments, the liquid formulation is used for oral administration, e.g., in a liquid-filled capsule or an oral solution.

### Capsule

In another aspect, there is provided a capsule comprising the pharmaceutical composition of described herein. In some embodiments, the capsule is a liquid fill capsule. In some embodiments, the liquid fill capsule comprises a liquid formulation of the pharmaceutical composition as described herein. In an embodiment, the liquid fill capsule has a volume from about 0.4 mL to about 0.9 mL, optionally from about 0.6 mL to about 0.8 mL, optionally about 0.7 mL. In an embodiment the capsule comprises any suitable outer shell. In an embodiment the capsule is a hard gelatin capsule or a soft gelatin capsule. In an embodiment the outer shell comprises any suitable material, such as gelatin or hypromellose.

In some embodiments, the concentration of the compound of Formula (I) in the liquid fill capsule is from 30 mg/mL to 100 mg/mL, or from 40 mg/mL to 80 mg/mL, or from 50 mg/mL to 75 mg/mL.

### Oral Solution

In another aspect, there is provide an oral solution comprising the pharmaceutical composition described herein. In an embodiment the oral solution comprises any liquid formulation of the pharmaceutical composition described herein further comprising sweeteners, common taste-masking agents, flavors and/or colors, the addition of which may make the composition more palatable. In an embodiment, the concentration of the active ingredient in the oral solution may be increased or decreased to allow measurement to be carried out in suitable manner depending on the conventional dosing devices used. Conventional dosing devices include a spoon, a dosing syringe, a dosing cup. The composition may be administered in the form of a medicine, a syrup, an elixir, syrup or a suspension.

### Injectable Solution

In another aspect, there is provided an injectable solution comprising the pharmaceutical composition described herein, which can be further diluted using standard intravenous infusion fluids to a target concentration of the active ingredient that is suitable for administration by intravenous infusion.

In some embodiments, the final infusate following dilution with a standard infusion fluid medium is also an injectable solution, which comprises a pharmaceutically acceptable solvent or intravenous fluid medium. In some embodiments, the pharmaceutically acceptable solvent or intravenous fluid medium may be selected from sterile water-for-injection, one or more hypotonic solution(s), 0.9% sodium chloride solution (Normal Saline), 0.45% sodium chloride solution (half-normal saline), 0.225% sodium chloride solution (quarter-normal saline) and/or dextrose solution, for example, 5% dextrose (DSVV). In some embodiments, the pharmaceutically acceptable solvent or intravenous fluid medium is an aqueous solution comprising 0.8 wt. % to about 1.0 wt. % sodium chloride, or about 0.9 wt. % sodium chloride.

In some embodiments, the pharmaceutical composition comprises a solubilizing agent. In some embodiments, the solubilizing agent is a complexing agent. The solubilizing agent may be selected from a polymer, a chelating agent, a counter-ion (e.g. suitable salt-forming counter ion) or a combination thereof. The surfactant and co-solvent may also be considered to be a solubilizing agent. In some embodiments, the solubilizing agent encapsulates the compound of Formula (I) and can form, for example, a liposome or a micelle.

In some embodiments, the solubilizing agent is a polymer. In some embodiments, the pharmaceutical composition comprises one, two, three, or four or more polymers. In some embodiments, the polymer may encapsulate the compound of Formula (I). In some embodiments, the polymer is selected from methyl acrylate-methacrylic acid copolymers, cellulose acetate phthalate, cellulose acetate succinate, hydroxypropyl methyl cellulose phthalate, hydroxypropyl methyl cellulose acetate succinate, polyvinyl acetate phthalate, shellac, cellulose acetate trimetallate, sodium alginate, zein, polyvinylpyrrolidone, poly(caprolactone) (PCL), poly(lactic-co-glycolic acid) (PLGA), poly(lactic acid) (PLA), (polyhydroxybutyrate) (PHB), poly(methysilsesquioxane) (PMSQ) or combinations thereof. In some embodiments, the polymer is a biodegradable (e.g. hydrolyzable) polymer, for example, PCL, PLGA, PLA or PHB. In some examples, the pharmaceutical composition comprises at least 0.1 wt. % polymer by weight, or at least 0.5 wt. %, or at least 1 wt. %, or at least 5 wt. %, or at least 10 wt. %, or at least 20 wt. % polymer by weight of the total pharmaceutical composition.

In some embodiments, the solubilizing agent is a suitable counter ion (e.g. to form a salt).

In some embodiments, the chelating agent may be selected from EDTA and salts thereof, citric acid, malic acid, malonic acid, oxalic acid, succinic acid, tartaric acid or a combination thereof.

In some embodiments the pharmaceutical composition comprises a wetting agent. In some embodiments, the wetting agent is a selected from benzalkonium chloride, poloxamers (e.g. poloxamer 188, poloxamer 407), polysorbate, sodium lauryl sulfate, hypromellose or a combination thereof.

In some embodiments the pharmaceutical composition comprises a solubilizing agent and a wetting agent. In some embodiment the pharmaceutical composition comprising a solubilizing agent in and amount of at least 0.1% (w/w) and a wetting agent in an amount of at least 0.1% (w/w).

In some examples the pharmaceutical composition comprises PEG in the amount of 30% (w/w) and hypromellose in an amount of 0.5% (w/w).

In some embodiments, the pharmaceutical composition is a compressed tablet. The compressed tablet comprises one or more compression aids and bulking agents, disintegrants, lubricants and wetting agents. The disintegrants may be selected from cross-linked carboxymethylcellulose (croscarmellose) sodium, carboxymethylcellulose calcium, carboxymethylcellulose sodium, sodium alginate, guar gum, cross-linked polyvinylpyrrolidone or crospovidone, cross-linked starch, sodium starch glycosylate, or any combination thereof. The disintegrants may increase the speed of release from the tablet and intestinal absorption of the compound of Formula (I). In some embodiments, the pharmaceutical composition is in the form of a tablet which comprises one or more disintegrants. In some examples, the pharmaceutical composition comprises at least 1 wt. % disintegrants by weight, or at least 5 wt. %, or at least 10 wt. %, or at least 20 wt. % disintegrants, by weight of the total pharmaceutical composition.

In some embodiments, the pharmaceutical composition is in the form of a tablet, wherein the tablet comprises a coating. In some embodiments, the coating comprises one or more of polyvinylalcohol, hydroxypropylmethocellulose, hydroxypropylcellulose, ethylcellulose, shellac, alginates, acrylate polymer, ferric oxide for color, or any combination thereof. In some embodiments the pharmaceutical composition is a direct compression tablet.

In some embodiments, the pharmaceutical composition comprises pharmacologically acceptable excipients selected from a filler, a glidant, a lubricant, an anti-oxidant, a mucolytic agent, a buffer, a pH adjuster, a tonicity adjuster, or a combinations thereof. These excipients may be additional to or equivalent to the ones described above.

In some embodiments, the filler may be selected from lactose, mannitol, sucrose, calcium sulphate, calcium phosphate, microcrystalline cellulose, xylitol, sorbitol, glucose, dextrose, mannose, maltitol or a combination thereof.

In some embodiments, the lubricant and glidant may independently be selected from a fatty acid, fatty acid salts, fatty acid monoglycerides, fatty acid triglycerides, fatty acid esters, talc, silica (for example, colloidal silica), or a combination thereof. The fatty acids may be a saturated or unsaturated fatty acid. The fatty acid may be a C₁₀-C₂₂ fatty acid. In some examples, the lubricant is selected from stearic acid, magnesium stearate, sodium behenate and/or sodium stearyl fumarate.

In some embodiments, the anti-oxidant is selected from ascorbic acid, citric acid, sodium citrate, vitamin A, vitamin E, cysteine hydrochloride, methionine or a combination thereof.

In some embodiments, the buffer may be selected from hydrochloric acid, sodium hydroxide tris, acetate, citrate, tartaric acid or salts thereof, lactic acid and salts thereof, phosphates, benzoates, bicarbonate, carbonates, sulphates, sodium chloride, potassium chloride, calcium chloride, tromethamine or a combination thereof. In some embodiments, the buffer may be tris. The buffer may be included to improve the stability of the Compound of Formula (I), e.g., Remdesivir. In some embodiments, the pharmaceutical composition may comprise up to about 5% buffer by weight of the pharmaceutical composition, or up to about 2% buffer by weight of the pharmaceutical composition, or from about 0.5 to about 1% buffer by weight of the pharmaceutical composition. In some embodiments, the pharmaceutical composition may comprise up to about 2% tris by weight of the pharmaceutical composition, or from about 0.5 to about 1% tris by weight of the pharmaceutical composition.

In some embodiments, the pH adjuster may be selected from hydroxides (e.g. sodium, magnesium, calcium, potassium), metal oxides (e.g., magnesium, calcium) acetic acid or salts thereof, citric acid or salts thereof, tartaric acid or salts thereof, lactic acid and salts thereof, gluconic acid and salts thereof, phosphates, pyrophosphates, benzoates, bicarbonate, carbonates, sulphates, sodium chloride, potassium chloride or a combination thereof, meglumine, adipic acid or salts thereof, tartaric acid or salts thereof, fumaric acid or salts thereof, gluconic acid or salts thereof, itaconic acids or salts thereof, ammonium aluminium sulfate, ammonium bicarbonate, ammonium hydroxide.

In some embodiments, the pH of the pharmaceutical composition in solution or as a suspension is from 1 to 11. In some embodiments, the pH of the pharmaceutical composition is slightly basic, for example, from 7.5 to 8. In some embodiments the pH of the pharmaceutical composition is slightly acidic, for example, less than 7, from 1 to <7, of from 1.5 to 6.75, or from 2 to 6.5, or from 4 to 6.75, or from 4 to 6.5, or from 4 to <7, or from 5 to 6.5, or from 5 to <7, or from 6 to <7, or from 6.5 to <7, or from 3 to 6, 4 to 6, 5 to 6, or 3 to 5, or 4 to 5, or 3 to 4. The pH of the pharmaceutical composition can alter the solubility and promote dissolution of the compound of Formula (I) and/or metabolites. In some embodiments, an acidic pH promotes the dissolution of a compound of Formula (I).

In some embodiments, the mucolytic agent may be N-acetyl cysteine or cysteine hydrochloride.

In some embodiments, the tonicity adjuster may be selected from dextrose, glycerin, mannitol, potassium chloride, sodium chloride or a combination thereof.

The pharmaceutical composition described herein may comprise from 10 mg to 1000 mg of Formula (I), or from about 100 mg to 1000 mg, or from about 20 mg to about 300 mg, or about 100 mg to 200 mg. The pharmaceutical composition described herein may comprise greater than 50 mg, or greater than 100 mg, or greater than 150 mg, or greater than 200 mg, or greater than 250 mg, or greater than 300 mg, or greater than 350 mg, or greater than 400 mg, or greater than 500 mg, or greater than 550 mg, or greater than 600 mg of Formula (I). In some cases, the pharmaceutical composition may comprise less than 1000 mg, or less than 500 mg, or less than 200 mg. In some cases, the oral dosage of Formula (I) may be larger than that used for intravenous injection because the mode of administration is different. The oral dosage may comprise one or more tablets, for example, two tablets, three tablets, or four tablets. The oral dosage may comprise one or more capsules, for example, two capsules, three capsules, or four capsules.

The pharmaceutical compositions described herein may have good shelf-life and/or stability. The stability of the pharmaceutical composition of the present invention may be monitored using a number of methods. The stability may be determined by establishing the initial amount of compound of Formula (I), and then measuring the amount of compound of Formula (I) remaining after a certain time thereafter and comparing the two values. The initial amount of the compound of Formula (I) is the amount present immediately after mixing all the components of the composition. The amount of compound of Formula (I) present may be measured using a range of methods known in the art, such as HPLC, mass spectrometry, spectrophotometry, gel electrophoresis, Western Blotting, light scattering, microbiological or other biological activity measuring assays. A typical method of tracking stability would constitute comparing the purity of the compound of Formula (I) in a given product pharmaceutical composition against that of a freshly prepared standard to calculate the amount of non-degraded compound of Formula (I) in the product for any given sample. Samples that are stored and analyzed over various periods of time would then provide a quantitative profile of the purity of the compound of Formula (I) over time. Optionally, the degradation rate of the compound of Formula (I) under stressed conditions of storage, such as at an elevated temperature, can then be determined from the decreasing purity versus time profile by fitting suitable regression lines or curves. Such degradation rates generated from stressed stability studies are particularly useful in comparing between different product pharmaceutical compositions over a short period of time.

In certain embodiments, at least 90% by weight of the compound of Formula (I) is present in the pharmaceutical composition after being stored for 30 days at from about 20° C. to about 25° C., based on the initial amount of the compound of Formula (I) in the pharmaceutical composition. For example, at least 92%, or at least 94%, or at least 96%, or at least 98% of the compound of Formula (I) is present in the composition after being stored for 30 days at from about 20 to about 25° C., based on the initial amount of the compound of Formula (I) in the pharmaceutical composition. In some examples, the composition is stored at about 25° C., or at about 24° C., or at about 23° C., or at about 22° C., or at about 21° C. The purity of the composition according to the present invention may be monitored using one or more analytical methods from those listed before that are most suited for compound of Formula (I) in question. The loss in purity may be determined by subtracting the purity of the compound of Formula (I) in the product at any given time from that immediately after manufacturing of the product (time to). The difference in purities would constitute the loss of purity over the time period of testing. Alternatively, the purity of the compound of Formula (I) could be measured at various time points from samples that are manufactured and stored in suitable sealed containers, which represent the unit dosage form. The purities are then plotted against time and fitted to a regression line, if linear, to determine an overall pseudo first-order degradation rate from the slope of such regression line.

The pharmaceutical composition described herein has good bioavailability. In some embodiments, the pharmaceutical composition described herein has good bioavailability when administered orally. In some embodiments, the bioavailability is at least 2.5%, or at least 3%, or at least 3.5%, or at least 4%, or at least 4.5%, or at least 5%, or at least 5.5%, or at least 6%, or at least 6.5%, or at least 7%, or at least 7.5%, or at least 8%, or at least 8.5%, or at least 9%, or at least 9.5%, or at least 10%, or at least 12.5%, or at least 15%, or at least 20%, or at least 25%, or at least 30%, or at least 35%, or at least 40%, or at least 45%, or at least 50% of the active ingredient or a metabolite formed in the body as a consequence of action of enzymes on the active ingredient as compared to when administered intravenously. In this case, the oral bioavailability is defined as AUC [(oral)/AUC(IV)]*100.

### Method of Treating a Viral Infection

In a second aspect, there is provided a method of treating a viral infection, the method comprising administering to a subject in need thereof a therapeutically effective amount any pharmaceutical composition described herein. In some embodiments, the viral infection is an RNA viral infection.

In some embodiments, virus causing the viral infection is a human-disease causing virus. In some embodiments, the virus may be coronavirus, respiratory syncytial virus, ebola, hepatitis, junin, lassa fever, orthomyxovirus, Hepatitis Virus (HV) type, disease-causing picornavirus, Ebola, SARS, MERS, respiratory syncytial virus and other pneumovirus, influenza, polio measles and retrovirus including adult Human T-cell lymphotropic virus type 1 (HTLV-1) and human immunodeficiency virus (HIV).

In some embodiments, the RNA virus may be a coronavirus. In some embodiments, the coronavirus is a coronavirus causing disease in humans. In some embodiments, the virus may be a coronavirus that causes disease in non-human animal species, such as, the feline infectious peritonitis virus, the porcine deltacorona virus.

In some embodiments, the viral infection is a coronavirus infection. The coronavirus infection may be caused by any type or strain of coronavirus. In some embodiments, the coronavirus infection may be an alphacoronavirus infection or a betacoronavirus infection, preferably a betacoronavirus. The betacoronavirus may have an A lineage, a B lineage, a C lineage or a D lineage, for example, a B lineage. In preferred embodiments, the coronavirus infection may be COVID-19, otherwise known as SARS-CoV-2 or 2019-nCoV.

In some embodiments, the pharmaceutical composition described herein may be administered in circumstances where there is an anticipated risk of infection for prophylactic use to prevent such infection or, at the very least, to prevent severe manifestations of the disease.

In some embodiments, the pharmaceutical composition may be administered using any suitable administration method. In an embodiment, the pharmaceutical composition is administered orally, parenterally, by inhalation or by nebulisation, or by intratracheal instillation. In an embodiment, the pharmaceutical composition is administered such that there is targeted delivery of the Compound of Formula (I) to the site of viral infection, e.g., in the case of SARS-CoV-2 to the lung, for example, to lung alveolar cells. Targeted drug delivery refers to any method of drug delivery that increases the concentration of the medication in some parts of the body relative to the others. In some embodiments, the pharmaceutical composition may be administered by injection. In some embodiments, the injection may be an intravenous injection or a subcutaneous injection. In some instances, subcutaneous injection can be advantageous because it is often non-intrusive, safe, well-tolerated, and/or requires reduced resource use due to reduced need for specialized skills or monitoring during administration.

In some embodiments, the pharmaceutical composition may be administered orally.

In some embodiments, the pharmaceutical composition described herein may be administered less than 4 days after exposure to a COVID-19 case (e.g. another positive case), or less than 3 days after exposure, or less than 2 days after exposure, or within a day of exposure, or within an hour of exposure to a COVID-19 case.

In some embodiments, the pharmaceutical composition described herein may be administered to prevent a possible viral exposure to an otherwise healthy individual without any notable signs or symptoms of the disease primarily for the purpose of prophylactic prevention of infection.

In some embodiments, the pharmaceutical composition described herein may alleviate one or more of the following symptoms caused by COVID-19: cough, sore throat, a high temperature or fever, loss of smell or taste, difficulty in breathing, tiredness, muscle pain, chest pain, runny nose, headache, chills, or any combination thereof.

In some embodiments, the viral infection is a hepatitis infection. The hepatitis infection may be hepatitis A, B, C, D or E. The hepatitis infection may be acute hepatitis, fulminant hepatitis or chronic hepatitis.

In some embodiments, the pharmaceutical composition described herein is administered from every 4 hours up to every 4 weeks. In an embodiment, the pharmaceutical composition described herein is administered every 4 hours, or up to every 8 hours, or up to every 12 hours, or up to every 16 hours, or up to every 24 hours, or up to every 48 hours, or up to every 36 hours, or up to every 72 hours, or up to every 144 hours, or up to every week, or up to every 2 weeks, or up to every 4 weeks. The compound of Formula (I) may be administered more frequently if the symptoms are more severe. An effective amount of the pharmaceutical composition described herein may be administered in either single or multiple doses. The multiple doses may be taken at the same time, or at different timepoints in the day (e.g. once, twice, three times, four times, five times or even six times a day).

The amount of the compound to be administered (i.e. the dosage) is dependent on the specific viral infection being treated, the mammal being treated, the severity of the disorder or condition, the rate of administration, the disposition of the compound, the bioavailability of the specific compound, and its effective inhibitory concentration (IC₅₀) against the specific virus infection being treated. In an embodiment, the dosage of the compound of Formula (I) is from about 2 to 20 mg/kg, for example, 3 to 18 mg/kg, 5 to 15 mg/kg, 7 to 14 mg/kg or 10 to 12 mg/kg.

In some embodiments, the pharmaceutical composition is a liquid formulation. In some embodiments the amount of liquid formulation administered is about 1 mL to about 40 mL of liquid formulation, for example about 3 mL to about 35 mL, or about 5 mL to about 30 mL, or about 10 mL to about 30 mL, or about 10 mL to about 25 mL, or about 15 mL to about 25 mL. In some embodiments the amount of compound of Formula (I) administered to the subject is from 10 to 1000 mg, or from 20 to 300 mg, or from 100 to 200 mg of compound of Formula (I). In some embodiments, the liquid formulation is administered as a liquid-filled capsule as described herein. In some embodiments, more than one liquid filled capsule is administered to the subject, or more than two, or more than three, or more than four liquid filled capsules are administered to the subject.

In some embodiments of the method, the compound of Formula (I) is metabolized to the active metabolite (triphosphate—Compound (D)) such that the active metabolite is present at a concentration of at least 0.02 μM, or at least 0.04 μM, or at least 0.06 μM, or at least 0.08 μM, or at least 0.1 μM, or at least 0.15 μM in the peripheral blood cells and/or target tissues.

In some embodiments of the method, the compound of Formula (I) is metabolized to the active metabolite (triphosphate—Compound (D)) such that the peak concentration of the active metabolite is at least 0.5 μM, or at least 0.7 μM, or at least 1 μM, or at least 10 μM in the peripheral blood cells and/or target tissues.

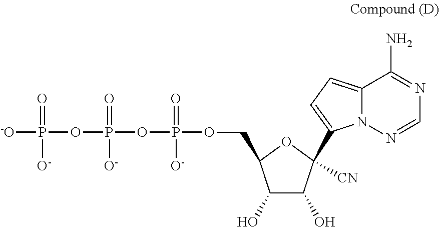

In some embodiments of the method, the bioavailability of the compound of Formula (I) after administration as measured in terms of the compound of Formula (I) or any of its direct metabolites in the blood, such as, Compound (C) [GS-441524] is at least 3%, or at least 5% or at least 7%, or at least 10%, or at least 50%, or at least 80%, or at least 90% as measured by the area under the plasma concentration versus time curve (AUC).

In some embodiments of the method, the bioavailability of Formula (I) inside peripheral blood cells, when measured in terms of Compound (D), after administration, is at least 3%, or at least 5% or at least 7%, or at least 10%, or at least 50%, or at least 80% as measured by the area under the concentration in peripheral blood cells versus time curve (AUC).

In some embodiments of the method, the bioavailability of the compound of Formula (I) after oral administration is at least 2.5%, or at least 3%, or at least 3.5%, or at least 4%, or at least 4.5%, or at least 5%, or at least 5.5%, or at least 6%, or at least 6.5%, or at least 7%, or at least 7.5%, or at least 8%, or at least 8.5%, or at least 9%, or at least 9.5%, or at least 10%, or at least 12.5%, or at least 15%, or at least 20%, or at least 25%, or at least 30%, or at least 35%, or at least 40%, or at least 45%, or at least 50 as compared to intravenous administration.

In some embodiments of the method, the oral bioavailability of Compound (C) (i.e. the nucleoside analog GS-441524) as measured with respect to the intravenous dose of the compound of Formula (I), and as measured from the concentration of Compound (C) in the blood after oral administration of compound of Formula (I) is at least 2.5%, or at least 3%, or at least 3.5%, or at least 4%, or at least 4.5%, or at least 5%, or at least 5.5%, or at least 6%, or at least 6.5%, or at least 7%, or at least 7.5%, or at least 8%, or at least 8.5%, or at least 9%, or at least 9.5%, or at least 10%, or at least 12.5%, or at least 15%, or at least 20%, or at least 25%, or at least 30%, or at least 35%, or at least 40%, or at least 45%, or at least 50% or at least 60%, or at least 70%, or at least 80% as compared to intravenous administration of the compound of Formula (I).

In some embodiments of the method, the oral bioavailability of Compound (D) (i.e. the active triphosphate) as measured with respect to the intravenous dose of the compound of Formula (I), and as measured from the concentration of Compound (D) in peripheral blood cells after oral administration of compound of Formula (I) is at least 2.5%, or at least 3%, or at least 3.5%, or at least 4%, or at least 4.5%, or at least 5%, or at least 5.5%, or at least 6%, or at least 6.5%, or at least 7%, or at least 7.5%, or at least 8%, or at least 8.5%, or at least 9%, or at least 9.5%, or at least 10%, or at least 12.5%, or at least 15%, or at least 20%, or at least 25%, or at least 30%, or at least 35%, or at least 40%, or at least 45%, or at least 50 as compared to intravenous administration of the compound of Formula (I).

The present disclosure may be described by one or more of the following paragraphs:
A. An oral formulation comprising a compound of Formula (I)

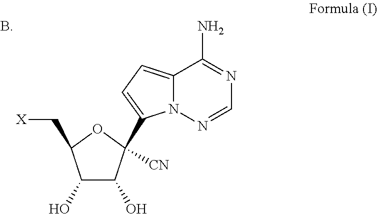

wherein X is selected from a hydroxyl, a metal salt hydroxylate, an O-linked phosphoester, an O-linked phosphoramidite, an O-linked ester, an O-linked carbamate, an S-linked phosphothioate, or an N-linked phosphoramidite.
B. The oral formulation of paragraph A, wherein the compound of Formula (I) is a prodrug.
C. The oral formulation according to paragraph A, wherein X is an O-linked phosphoramidite or an O-linked phosphoester.
D. The oral formulation according to any of the preceding paragraphs, wherein X is an O-linked phosphoramidite with formula

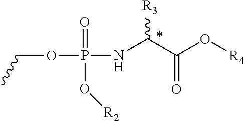

wherein R₂ is H, lithium, sodium, potassium, aluminium, ammonium, arginine benzathine, calcium, chloroprocaine, choline, diethanolamine, ethanolamine, ethylenediamine, lysine, magnesium, histidine, tromethamine, meglumine, procaine, trimethylamine, zinc, C₁-C₆ alkyl, C₂-C₆-allyl, C₂-C₆ alkenyl, phenyl, biphenyl, heteroaryl,
R₃ is H or C₁-C₆ alkyl; and
R₄ is H, lithium, sodium, potassium, aluminium, ammonium, arginine benzathine, calcium, chloroprocaine, choline, diethanolamine, ethanolamine, ethylenediamine, lysine, magnesium, histidine, tromethamine, meglumine, procaine, trimethylamine, zinc, C₁-C₆ alkyl, C₂-C₆-allyl, C₂-C₆ alkenyl, phenyl, biphenyl, heteroaryl,
E. The oral formulation according to paragraph D, wherein R₃ is methyl.
F. The oral formulation according to any one of paragraphs A to C, wherein X is an O-linked phosphoramidite with formula

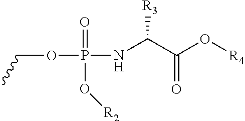

wherein R₂ is H, lithium, sodium, potassium, aluminium, ammonium, arginine benzathine, calcium, chloroprocaine, choline, diethanolamine, ethanolamine, ethylenediamine, lysine, magnesium, histidine, tromethamine, meglumine, procaine, trimethylamine, zinc, C₁-C₆ alkyl, C₂-C₆-allyl, C₂-C₆ alkenyl, phenyl, biphenyl, heteroaryl,
R₃ is H, methyl, ethyl, isopropyl, isobutyl, secbutyl, phenyl,
R₄ is H, lithium, sodium, potassium, aluminium, ammonium, arginine benzathine, calcium, chloroprocaine, choline, diethanolamine, ethanolamine, ethylenediamine, lysine, magnesium, histidine, tromethamine, meglumine, procaine, trimethylamine, zinc, C₁-C₆ alkyl, C₂-C₆-allyl, C₂-C₆ alkenyl, phenyl, biphenyl, heteroaryl.
G. The oral formulation according to any of the preceding paragraphs, wherein the compound of Formula (I) is

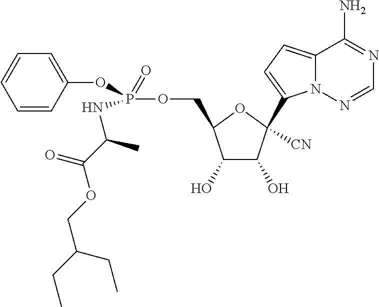 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

H. The oral formulation according to paragraph A, wherein X is a hydroxyl, a metal salt hydroxylate, an O-linked ester or an O-linked carbamate.
I. The oral formulation according to paragraph A or paragraph H, wherein the O-linked ester is an O-linked amino acid or an O-linked peptide.
J. The oral formulation according to paragraph I, wherein the O-linked amino acid or 0-linked peptide is formed from amino acids selected from alanine, valine, leucine, isoleucine, glycine, isoleucine, tyrosine, tryptophan or a combination thereof.
K. The oral formulation according to any preceding paragraph, wherein the compound of Formula (I) has a log P less than 2.
L. The oral formulation according to any preceding paragraph, wherein the compound of Formula (I) has a log S greater than −2.
M. The oral formulation according to any one of the preceding paragraphs, wherein the metabolised active product of compound of Formula (I) is Compound (D)

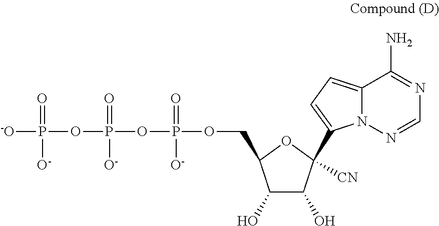

N. The oral formulation according to any one of the preceding paragraphs, wherein the oral formulation is in the form of a solid oral dosage form, a liquid oral dosage form, a capsule, a tablet, a liquid-filled capsule, a caplet, a chewable gum, an oral film, an oral solution, a suspension, an emulsion, a lozenge, a wafer, a granulated powder formulation, a simple powder or mixture thereof, an elixir or a syrup.
O. The oral formulation according to any one of the preceding paragraphs that is free from cyclodextrins.
P. The oral formulation according to any one of the preceding paragraphs, further comprising a solubilizing agent.
Q. The oral formulation according to paragraph P, wherein the solubilizing agent encapsulates the compound of Formula (I).
R. The oral formulation according to any one of paragraphs P or Q wherein the solubilizing agent is selected from a liposome, a micelle, a polymer, a surfactant, a co-solvent, a chelating agent, a counter-ion or a combination thereof.
S. The oral formulation according to paragraph R, wherein the solubilizing agent is a polymer, and wherein the polymer is selected from methyl acrylate-methacrylic acid copolymers, cellulose acetate phthalate, cellulose acetate succinate, hydroxypropyl methyl cellulose phthalate, hydroxypropyl methyl cellulose acetate succinate, polyvinyl acetate phthalate, shellac, cellulose acetate trimetallate, sodium alginate, zein, polyvinylpyrrolidone, poly(caprolactone), poly(lactic-co-glycolic acid), poly(lactic acid), poly(hydroxybutyrate), poly(methylsilsesquioxane) or a combination thereof.
T. The oral formulation according to paragraph S, wherein the polymer is a biodegradable polymer, optionally selected from poly(caprolactone), poly(lactic-co-glycolic acid), poly(lactic acid) and/or poly(hydroxybutyrate).
U. The oral formulation according to paragraph R, wherein the solublising agent is a co-solvent, and wherein the co-solvent is selected from PEG, glycerol, glycofural, DMSO, ethanol, propylene glycol, methyl lactate, ethyl lactate, propyl lactate, spironolactone, N-methylpyrrolidone, benzyl alcohol, cetostearyl alcohol, benzylbenzoate, corn syrup, acacia syrup, glucose syrup, acetyltributyl citrate, lactic acid, acetic acid, ethylacetate, benzoic acid, polyoxyl 35 castor oil, polysorbate 20, 40, and 80; water; mineral oils, edible hydrogenated oils; edible non-hydrogenated edible oils or a combination thereof.
V. The oral formulation according to paragraph R, wherein the solubilising agent is a counter-ion, wherein the counter-ion is a metal ion, and wherein the metal-ion is selected from Ag⁺, Fe²⁺, Fe³⁺, Co²⁺, Co³⁺, Cu²⁺, Zn²⁺, or a combination thereof.
W. The oral formulation according to paragraph R, wherein the solubilizing agent is a chelating agent, and wherein the chelating agent is selected from EDTA and salts thereof, citric acid, malic acid, malonic acid, oxalic acid, succinic acid, tartaric acid or a combination thereof.
X. The oral formulation according any of the preceding paragraphs, further comprising one or more wetting agent selected from benzalkonium chloride, poloxamers (e.g. poloxamer 188, poloxamer 407), polysorbate, sodium lauryl sulfate, hypromellose or a combination thereof.
Y. The oral formulation according any of the preceding paragraphs, further comprising one or more disintegrants selected from cross-linked carboxymethylcellulose (croscarmellose) sodium, carboxymethylcellulose calcium, carboxymethylcellulose sodium, sodium alginate, guar gum, cross-linked polyvinylpyrrolidone or crospovidone, cross-linked starch, sodium starch glycosylate, or any combination thereof,
Z. The oral formulation any of the preceding paragraphs, wherein the oral formulation is a tablet, and wherein the tablet comprises a coating comprising one or more of polyvinylalcohol, hydroxypropylmethocellulose, hydroxypropylcellulose, ethylcellulose, shellac, alginates, acrylate polymer, ferric oxide or a combination thereof.
AA. The oral formulation according to any one of the preceding paragraphs further comprising one or more pharmacologically acceptable excipients selected from a filler, a glidant, a lubricant, an anti-oxidant, a mucolytic agent, a buffer, a pH adjuster, a tonicity adjuster, or a combination thereof.
BB. The oral formulating according to paragraph AA, wherein the filler is selected from lactose, mannitol, sucrose, calcium sulphate, calcium phosphate, microcrystalline cellulose, xylitol, sorbitol, glucose, dextrose, mannose, maltitol or a combination thereof.
CC. The oral formulation according to paragraph AA, wherein the lubricant is selected from a vegetable oil, an animal oil, a fatty acid, fatty acid salts, fatty acid monoglycerides, fatty acid triglycerides, talc, silica, or a combination thereof.
DD. The oral formulation according to paragraph AA, wherein the anti-oxidant is selected from ascorbic acid, citric acid, sodium citrate, vitamin A, vitamin E, cysteine hydrochloride, methionine or a combination thereof.
EE. The oral formulation according to any one the preceding paragraphs comprising 10 mg to 1000 mg of the compound of Formula (I), or from about 100 mg to 1000 mg of the compound of Formula (I).
FF. A method of treating a RNA viral infection, the method comprising orally administering to a subject in need thereof a therapeutically effective amount the oral formulation of any one of paragraphs A-Z or AA-EE.
GG. The method according to paragraph FF, wherein the viral infection is a coronavirus infection.
HH. The method according to paragraph FF, wherein the coronavirus infection is COVID-19.
II. The method according to paragraph FF, wherein the viral infection is a hepatitis infection.
JJ. The method according to any one of paragraphs FF to II, wherein the oral formulation is administered from every 4 hours up to every 4 weeks.
KK. The method according to any one of paragraphs FF to JJ, wherein the compound of Formula (I) is metabolized to the active metabolite of Compound (D) such that the active metabolite is present at a concentration of at least 0.1 μM in the peripheral blood cells and/or target tissues.

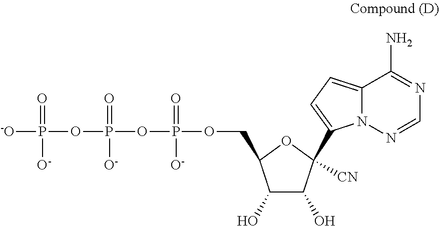

LL. The method according to any one of paragraphs FF to KK, wherein the bioavailability of the compound of Formula (I) is at least 5% after oral administration.

The present disclosure may also be described by one or more of the following paragraphs:
a. A pharmaceutical composition comprising a compound of Formula (I)

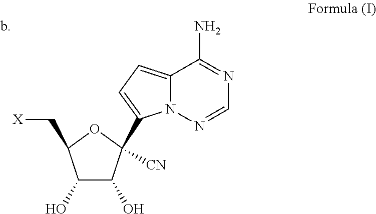

wherein X is selected from a hydroxyl, a metal salt hydroxylate, an O-linked phosphoester, an O-linked phosphoramidite, an O-linked ester, an O-linked carbamate, an S-linked phosphothioate, or an N-linked phosphoramidite, and at least one pharmaceutically acceptable excipient selected from a cysteine compound, an amino acid, an amino acid salt, an N-acetyl amino acid, an organic acid or a salt thereof, or any combination thereof.
b. The pharmaceutical composition according to paragraph a, wherein the pharmaceutical composition is free of cyclodextrin.
c. The pharmaceutical composition according to paragraph a or paragraph b, wherein the composition is a solution, a suspension or a mixture thereof.
d. The pharmaceutical composition according to any preceding paragraph, wherein the pharmaceutical composition is an oral formulation or a parenteral formulation.
e. The pharmaceutical composition according to any preceding paragraph, wherein the at least one pharmaceutically acceptable excipient comprises an acid or a salt thereof, and optionally wherein the acid is an organic acid selected from lactic acid, acetic acid, adipic acid, citric acid, formic acid, succinic acid oxalic acid, ascorbic acid, uric acid, malic acid, tartaric acid or any combination thereof.
f. The pharmaceutical composition according to any preceding paragraph, wherein the at least one pharmaceutically acceptable excipient comprises at least one cysteine compound.
g. The pharmaceutical composition according to paragraph f, wherein the % w/w ratio of the at least one cysteine compound to the compound of Formula (I) is at least 1:1, optionally greater than 1.5:1.
h. The pharmaceutical composition according to any one of paragraphs f-g, comprising
   a. 0.05-20% w/w compound of Formula (I)
   b. 0.5-50% w/w of at least one cysteine compound.
i. The pharmaceutical composition according to any one of paragraphs f-h, comprising
   a. 4-8% w/w compound of Formula (I), optionally 5-7% w/w compound of Formula (I) and
   b. 1-30% w/w of at least one cysteine compound.
j. The pharmaceutical composition according to any one of paragraphs f-i, wherein the at least one cysteine compound comprises cysteine, glutathione, cysteine hydrochloride and/or N-acetyl cysteine or a combination thereof.
k. The pharmaceutical composition according to any one of paragraphs f-j, wherein the at least one cysteine compound is cysteine hydrochloride and/or N-acetyl cysteine.
l. The pharmaceutical composition according to any one of paragraphs f-k, wherein the at least one cysteine compound is cysteine hydrochloride and N-acetyl cysteine.
m. The pharmaceutical composition according to any one of paragraphs f-l comprising
   a. 3-10% w/w of compound of Formula (I)
   b. 0.5-15% w/w cysteine hydrochloride monohydrate
   c. 0.5-15% w/w N-acetyl cysteine.
n. The pharmaceutical composition according to any preceding paragraph, wherein the pharmaceutical composition is a liquid formulation.
o. The pharmaceutical composition according to paragraph n, wherein the pharmaceutical composition comprises one or more co-solvents.
p. The pharmaceutical composition according to any one of paragraphs n-o, wherein the liquid formulation has a pH of less than 8.5, optionally wherein the pH is in the range of 1-<8.
q. The pharmaceutical composition according to any one of paragraphs n-p comprising
   a. 3-10% w/w of compound of Formula (I)
   b. 0.5-30% w/w a cysteine compound
   c. 50-86% w/w of one or more co-solvent.
r. The pharmaceutical composition according to paragraphs n-q comprising
   a. 3-10% w/w of compound of Formula (I)
   b. 1-15% w/w cysteine hydrochloride monohydrate,
   c. 3-15% w/w N-acetyl cysteine
   d. 50-86% w/w one or more co-solvent.
s. The pharmaceutical composition according to paragraph n-r, wherein the one or more co-solvents is selected from PEG, benzyl alcohol, ethanol or a combination thereof.
t. The pharmaceutical composition according to paragraph n-s, wherein the one or more co-solvents comprises low molecular weight polyethylene glycols (PEG), propylene glycol, benzyl alcohol, ethanol or a combination thereof.
u. The pharmaceutical composition according to paragraph t, wherein the PEG has a molecular weight from 200 to 1000.
v. The pharmaceutical composition according to any one of the preceding paragraphs, comprising one or more surfactants.
w. The pharmaceutical composition according to paragraph v, wherein the one or more surfactants is selected from polysorbate 20, polysorbate 40, polysorbate 60, polysorbate 80, polyoxyl 35 castor oil, cremophor, polyoxyethylene (20) sorbitan monooleate, polyethylene glycol sorbitan monooleate, polyoxyethylenesorbitan monooleate, or a block copolymer of poly(ethylene oxide) (PEO) and poly(propylene oxide) (PPO), such as, poloxamer, or a combination thereof.
x. The pharmaceutical composition according to any one of paragraphs v-w, wherein the HLB value of the one or more surfactant is from 10-20, optionally from 12 to 18, or optionally from 14 to 16.
y. The pharmaceutical composition according to any one of paragraphs v-x, wherein the one or more surfactants is polysorbate.
z. The pharmaceutical composition according to any one of paragraphs v-y, comprising
   a. 3-10% w/w of compound of Formula (I)
   b. 1-15% w/w cysteine hydrochloride monohydrate
   c. 3-15% w/w N-acetyl cysteine
   d. 50-83% w/w one or more co-solvent
   e. 2-8% w/w surfactant.
aa. The pharmaceutical composition according to any one of paragraphs x-z comprising
   a. 3-10% w/w of compound of Formula (I)
   b. 1-15% w/w cysteine hydrochloride monohydrate
   c. 3-15% w/w N-acetyl cysteine
   d. 4-8% w/w polysorbate 80
   e. 35-60% w/w PEG 400
   f. 10-30% w/w PEG 300
   g. % w/w ethanol
   h. % w/w benzyl alcohol.
bb. The pharmaceutical composition according to any one of the preceding paragraphs, comprising one or more anti-oxidants.
cc. The pharmaceutical composition according to any one of the preceding paragraphs, wherein the metabolised active product of compound of Formula (I) is Compound (D)

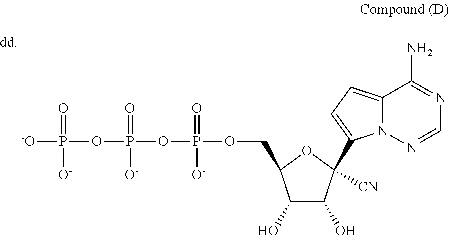

ee. The pharmaceutical composition according to any preceding paragraph, wherein the compound of Formula (I) is

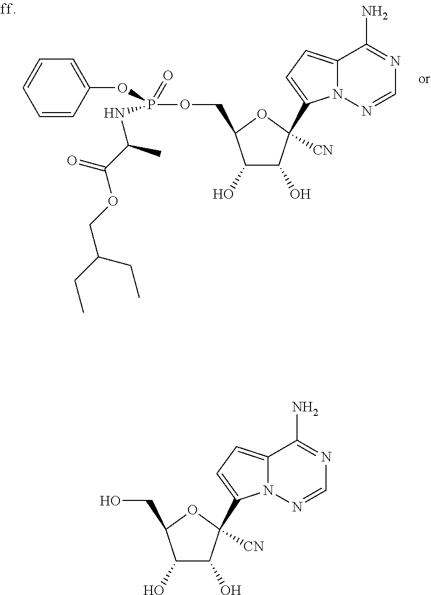 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1 ; N#C[C@@]1(c2ccc3c(N)ncnn23)O[C@H](CO)[C@@H](O)[C@H]1O]

gg. The pharmaceutical composition according to any preceding paragraph, wherein the pharmaceutical composition is formulated such that the compound of Formula (I) has a solubility of greater than 0.05 mg/mL when placed in an aqueous solution at pH 6.5.
hh. A method of treating a viral infection, the method comprising administering to a subject in need thereof a therapeutically effective amount of the pharmaceutical composition of any one of paragraphs a-gg.
ii. The method according to paragraph hh, wherein the virus causing the viral infection is selected from a coronavirus, respiratory syncytial virus, ebola, hepatitis, junin, lassa fever, orthomyxovirus, Hepatitis Virus (HV) type, disease-causing picornavirus, Ebola, SARS, MERS, respiratory syncytial virus and other pneumovirus, influenza, polio measles and retrovirus including adult Human T-cell lymphotropic virus type 1 (HTLV-1) and human immunodeficiency virus (HIV).
jj. The method according to paragraph hh, wherein the viral infection is a coronavirus infection.
kk. The method according to paragraph jj, wherein the coronavirus infection is SARS-CoV-2.
ll. The method according to any one of paragraphs hh-jj, wherein the amount of the compound of Formula (I) administered is from about 20 mg to about 300 mg, or from about 50 mg to 250 mg.
mm. The method according to any one of paragraphs hh-ll, wherein the pharmaceutical composition is a liquid formulation and optionally wherein the amount of liquid formulation dosed is from about 1 mL to about 40 mL of the undiluted liquid formulation.
nn. The method according to any one of paragraphs hh-mm, wherein the pharmaceutical composition is administered orally.
oo. The method according to any one of paragraphs hh-nn, wherein the pharmaceutical composition is administered by injection.
pp. The method for use according to paragraph oo, wherein the injection is an intravenous injection or a subcutaneous injection.
qq. The method for use according to paragraph pp, wherein the injection is an intravenous injection or a subcutaneous injection that is administered following dilution with an intravenous infusion fluid to a final infusate volume of 100 to 250 mL.
rr. A capsule comprising the pharmaceutical composition of any one of paragraphs a-gg.
ss. A capsule according to paragraph rr, wherein the capsule is a liquid fill capsule.
tt. A capsule according to paragraph ss, wherein the liquid fill capsule has a volume from about 0.4 mL to about 0.9 mL, optionally from about 0.6 mL to about 0.8 mL, optionally about 0.7 mL.
uu. An oral solution comprising the pharmaceutical composition of any one of paragraphs a-gg.
w. An injectable solution comprising the pharmaceutical composition of any one of paragraphs a-gg.

### Examples

### Effect of Pharmaceutical Excipients on Solubility

Various compounds were tested in combination with Remdesivir to see if the compounds had a solubilizing effect. The present inventors found that cysteine-compounds in particular (e.g. cysteine hydrochloride, N-acetyl cysteine, L-cysteine and glutathione) all demonstrated a solubilizing effect on Remdesivir, with solubility being greater >0.01 mg/mL for all cysteine-related compounds at concentrations that are suitable for administration. Furthermore, certain cysteine compounds, namely cysteine hydrochloride and N-acetyl cysteine were shown to improve Remdesivir solubility to an amount >0.2 mg/mL at concentrations of excipients that are suitable for administration by oral and parenteral routes. Without wishing to be bound by theory, in addition to any innate effect on solubility, the improvements of solubility of Remdesivir in solutions of cysteine hydrochloride and N-acetyl cysteine may be further attributed to a slight acidifying effect of these compounds.

N-acetyl amino acids, e.g. both N-acetyl cysteine and N-acetyl D-alanine, also showed a significant solubilizing effect.

TABLE 1
Pharmaceutical excipients and their effects on Remdesivir solubility
at concentrations of these excipients in water that may be justified
by the maximum daily intakes for each (MDI)†

| Excipient | Remdesivir Solubility (mg/mL) |
|---|---|
| DL-Tyrosine | 0.001 |
| Plasdone, PVP | 0.003 |
| L-Arginine | 0.000 |
| Tromethamine | 0.000 |
| Cysteine Hydrochloride | 0.546 |
| D-Sorbitol | 0.008 |
| Meglumine | 0.000 |
| Hypromellose | 0.004 |
| N-acetyl-D-alanine | 0.082 |
| Cavitron (HPβCD) | 0.050 |
| L-cysteine | 0.010 |
| N-acetyl-L-cysteine | 0.236 |
| Sodium sulfide | 0.000 |
| Sodium sulfate | 0.002 |
| Sodium sulfite | 0.000 |
| L-methionine | 0.000 |
| L-glutathione | 0.03 |
| Monothioglycerol | 0.004 |
| Saccharin sodium | 0.0005 |

†MDI or maximum daily intake is based on FDA's IIG database for approved oral dosage forms when diluted in 250 mL of water. This is to reflect final concentrations of these excipients that could be achieved in the intestine, if the maximum approved intake is administered along with the daily dose of the drug.

Due to the good results observed with cysteine HCl and N-acetyl cysteine, these were compared against sulfobutylether β cyclodextrin (SBEβCD), which is the known excipient used in Remdesivir formulations. The concentration of SBEβCD used was 11.8 mg/mL, which corresponds to approximately 3 g of SBEβCD dissolved in 250 mL of intestinal fluid which is approximately the same amount of SBEβCD (3 g) used for every 100 mg Remdesivir in the freeze-dried injectable formulation used commercially. As can be seen from Table 2, both cysteine hydrochloride and N-acetyl cysteine solubilized Remdesivir more effectively than SBEβCD on a weight basis. This indicates that the pharmaceutical compositions described herein may offer an alternative strategy of solubilizing and administering Remdesivir to improve its oral bioavailability, or for the intravenous administration of Remdesivir to patients with impaired kidneys, who may not be able to receive the current product containing high amount of SBEβCD.

TABLE 2
Comparison of Cysteine HCl and N-Acetyl-L-Cysteine
with Sulfobutylether β cyclodextrin

| | Concentration | Remdesivir |
|---|---|---|
| | in Water | Solubility |
| Excipient | (mg/mL) | (mg/mL) |
| Cysteine HCl | 20 | 0.91 |
| Cysteine HCl | 14 | 0.81 |
| Cysteine HCl | 9.3 | 0.55 |
| Cysteine HCl | 5 | 0.39 |
| N-Acetyl-L-Cysteine | 14 | 0.24 |
| N-Acetyl-L-Cysteine | 11 | 0.18 |
| Sulfobutylether β cyclodextrin | 11.8 | 0.05 |

### Effect of pH on Remdesivir Solubility

The solubility of Remdesivir was screened in a range of different acids to test the effect of pH and acidity on Remdesivir solubility. The results demonstrated in Table 3 indicate that the solubility of Remdesivir is increased at lower pH and in the presence of acids. This indicates that organic acids and other acidulants may improve the solubilization and dissolution of Remdesivir from an oral dosage form.

TABLE 3

| Solvent | pH (Range) | Solubility (mg/mL) |
|---|---|---|
| Hydrochloric acid solution | 0.98 | 2.49 |
| Hydrochloric acid solution | 1.38 | 0.98 |
| Hydrochloric acid solution | 1.89 | 0.34 |
| Hydrochloric acid solution | 2.91 | 0.005 |
| Hydrochloric acid solution | 4.74 | 0.0010 |
| Hydrochloric acid solution | 6.18 | 0.0009 |
| Hydrochloric acid solution | 6.72 | 0.00 |
| 1M Acetic acid solution | 1.5 | 0.32 |
| Boric acid solution | 3.5 | 0.01 |
| 1M Citric acid solution | 0.5-1.0 | 0.95 |
| 1M Ascorbic acid solution | 1.0-1.5 | 0.64 |
| 1M Tartaric acid solution | 0.5-1.0 | 1.69 |
| 1M Lactic acid solution | 1.5 | 0.74 |
| 1M Formic acid solution | 1.5 | 0.89 |

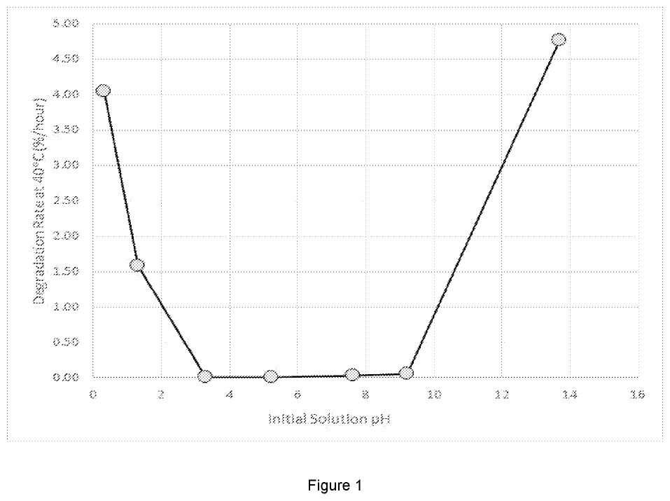

In order to determine the stability of the prodrug Remdesivir at different pH, the stability of prodrug Remdesivir was tested over time at physiologically relevant pH of gastrointestinal media. The degradation of Remdesivir was determined by measuring the amount of unchanged Remdesivir using a suitable high pressure liquid chromatographic (HPLC) method and comparing the results against a pure reference sample of Remdesivir. As shown in FIG. 1, the results show an acceptable stability and minimal degradation at a pH between 3 and 9.

### Effect of Co-Solvents on Solubility

Various co-solvents were screened for their solubilizing effect on Remdesivir, with results shown in Table 4.

TABLE 4

| Solvent | Solubility (mg/mL) |
|---|---|
| N-methyl-2-pyrrolidone (NMP) | ≥112 |
| Soluplus ® + Ethanol + Water | ≥34 |
| Plasdone (PVP) + PEG 300 | ≥66 |
| NMP + Ethanol | ≥226 |
| Ethanol | ≥21 |
| Benzyl Alcohol | ≥56 |
| Propylene Glycol | ≥9 |
| PEG 300 | ≥39 |
| PEG 400 | ≥16 |
| PEG 600 | ≥6.2 |
| PEG 1000 | ≥6.3 |
| PEG 400 + Glycerol (3:5) | 0.011 |
| PEG 400 + Glycerol (3:10) | 0.0008 |
| Castor Oil | 0 |
| Sesame Oil | 0 |
| Peppermint Oil | 15.9 |
| Soybean Oil | 0 |
| Peanut Oil | — |
| Mineral Oil | — |
| Span 80 | 0.3 |
| Span 20 | 0.006 |

The results demonstrated that solubilizing with suitable combinations of cosolvents and solubilizing excipients, it is possible to sustain the solubility of Remdesivir in various gastrointestinal media that would be encountered over the course of gastric transit and intestinal absorption following oral administration. Since only the dissolved drug substance is absorbed, the sustenance of solubility is critical for a poorly soluble and slow-dissolving molecule, such as, Remdesivir, for maximizing oral bioavailability. Similarly, the data demonstrates that certain combinations of cosolvents and solubilizing excipients may be useful for the formulation of an injectable dosage form that does not contain SBEβCD.

### Effect of Surfactants on Solubility

TABLE 5

| | | Concentration | Remdesivir |
|---|---|---|---|
| | HLB | in Water | Solubility |
| Surfactant | value | (% w/w) | (mg/mL) |
| TWEEN 20 (Polysorbate 20) | 16.7 | 0.16 | 0.016 |
| TWEEN 80 (Polysorbate 80) | ~15 | 0.30 | 0.033 |
| Poloxamer P188 | ~29 | 0.07 | 0.000 |
| Poloxamer P407 | ~18 | 0.07 | 0.006 |
| Kolliphor EL (Polyoxyl 35 | ~15 | 0.10 | 0.020 |
| Castor Oil, Cremophor) | | | |

Remdesivir solubility was also tested in the presence of various different surfactants, with polysorbate and polyoxyl castor oil surfactants (both with a HLB value of 15) demonstrating the best results.

### Pharmaceutical Compositions

Based in part on the solubility and stability studies at different pH cited above, the present inventors developed the following Example pharmaceutical compositions. Remdesivir was found to be effectively solubilized in these example pharmaceutical compositions. Furthermore, Example Pharmaceutical composition 1 showed good results in subsequent dissolution and solubility testing.

TABLE 6
Example Pharmaceutical composition 1 - ES040-36

| Ingredient | % (w/w) | Intake/100 mg dose | |
|---|---|---|---|
| Remdesivir | 7 | 100 | mg |
| Cysteine hydrochloride | 6 | 85.7 mg | |
| monohydrate | | (59.1 mg of Cysteine) | |
| N-acetyl-L-cysteine | 6 | 85.7 | mg |
| Tween 80 (Polysorbate 80) | 6 | 85.7 | mg |
| PEG 300 | 16 | 228.6 | mg |
| PEG 400 | 53 | 757.1 | mg |
| Ethanol | 4 | 78.3 | mg |
| Benzyl alcohol | 4 | 81.1 | mg |

TABLE 7
Example Pharmaceutical composition 2 - ES040-32A

| Ingredient | % (w/w) | Intake/100 mg dose |
|---|---|---|
| Remdesivir | 5 | 100 mg |
| Cysteine hydrochloride | 12 | 240 mg |
| monohydrate | | (165.6 mg of Cysteine) |
| N-acetyl-L-cysteine | 8 | 160 mg |
| Tween 80 | 6 | 120 mg |
| (i.e. Polysorbate 80) | | |
| PEG 300 | 20 | 400 mg |
| PEG 400 | 43 | 860 mg |
| Ethanol | 4 | 80 mg |
| Benzyl alcohol | 4 | 80 mg |

TABLE 8
Example Pharmaceutical composition 3 - ES040-86

| Ingredient | % (w/w) | Intake/100 mg dose | |
|---|---|---|---|
| Remdesivir | 6.7 | 100 | mg |
| Cysteine hydrochloride | 2.08 | 31.0 | mg |
| monohydrate | | | |
| N-acetyl-L-cysteine | 6.07 | 90.6 | mg |
| Tween 80 | 5.70 | 85.1 | mg |
| (i.e. Polysorbate 80) | | | |
| PEG 300 | 16.02 | 239.1 | mg |
| PEG 400 | 53.38 | 796.7 | mg |
| Ethanol | 4.07 | 60.7 | mg |
| Benzyl alcohol | 4.02 | 60 | mg |
| Tromethamine (Tris) | 0.71 | 10.6 | mg |
| Poloxamer P188 | 1.23 | 18.4 | mg |

TABLE 9
Example Pharmaceutical composition 4 - ES040-90

| Ingredient | % (w/w) | Intake/100 mg dose | |
|---|---|---|---|
| Remdesivir | 6.70 | 100 | mg |
| Cysteine hydrochloride | 3.33 | 49.7 | mg |
| monohydrate | | | |
| N-acetyl-L-cysteine | 6.22 | 92.8 | mg |
| Tween 80 | 6.01 | 89.7 | mg |
| (i.e. Polysorbate 80) | | | |
| PEG 300 | 16.46 | 245.7 | mg |
| PEG 400 | 55.03 | 821.3 | mg |
| Ethanol | 0 | 0 | mg |
| Benzyl alcohol | 4.11 | 61.3 | mg |
| Poloxamer P188 | 2.15 | 32.1 | mg |

### Dissolution and Solubility Study

The solubility and/or dissolution of Remdesivir over time was determined for various pharmaceutical compositions using a Type II dissolution apparatus. The dissolution of the liquid formulation filled into hard-gelatin capsule and “as-is”, i.e. without being filled into the capsule shell were compared to the release of the non-formulated powder drug substance filled into the capsule, as well as the latter in combination with the solubilizing complexing agent, SBEβCD. The dissolution profiles were compared to that of acetaminophen drug substance filled into a hard-gelatin capsule. Acetaminophen has over 80% bioavailability in practice. The dissolution tests were carried out at a stirring speed of 100 rpm with 15 mg Remdesivir in 300 mL of dissolution media comprising 0.4% solution of Tween 80 in water, which had been previously determined to provide enough solubility of Remdesivir for the test dose of 15 mg. At appropriate time intervals, about 1 mL aliquots of the dissolution media were withdrawn, filtered and tested using a high-pressure chromatographic assay method for Remdesivir. The area under the peak for the drug at the standard elution time was then compared against that of an external reference standard of Remdesivir to determine the concentration of dissolved Remdesivir at any given time.

Specifically, the Example formulation 1 (ES040-36) comprising Remdesivir was tested both as a solution added directly into the dissolution medium without being filled into a capsule shell to represent the release from an oral solution and also as filled into a hard-gelatin capsule. The Example formulations were compared to the solid form of the Remdesivir in a capsule containing no excipients or in the presence of the solubilizing excipient, SBEβCD, used in the currently marketed injected formulations, as a reference Example.

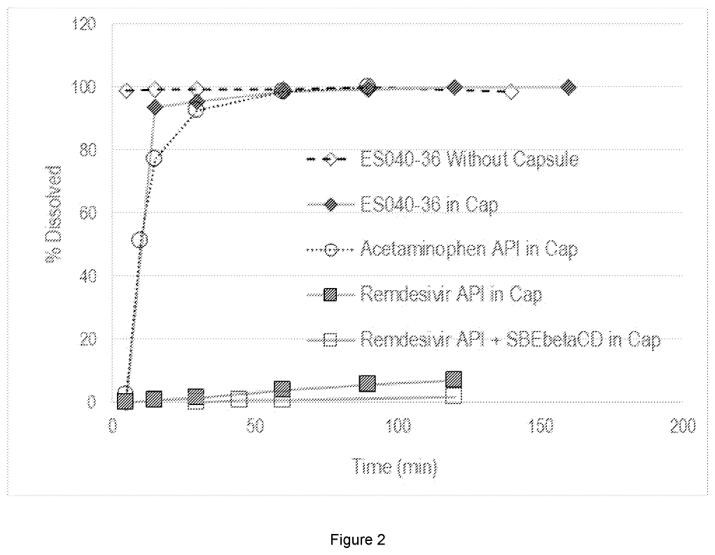

The results of the study are shown in FIG. 2. The diamond datapoints correspond to the pharmaceutical composition of the invention. The open diamonds with a dashed line represent the dissolution of the composition when it is directly added to the dissolution medium as a liquid phase without being filled into a hard-gelatin capsule; the filled diamonds with a solid line represent the dissolution of the composition when it has been filled into hard-gelatin capsule and then added to the dissolution medium. The circle datapoints with thin dashed line correspond to a different active ingredient, acetaminophen, to represent the dissolution profile of a drug with high bioavailability for comparison. The comparative control comprises powdered bulk acetaminophen drug substance filled into hard-gelatin capsule. The square datapoints represent the dissolution of Remdesivir drug substance as a powder filled into hard-gelatin capsule either with a solubilizing excipient, SBEβCD (open squares) or without any other excipient (closed squares).

In the absence of pharmaceutical excipients, Remdesivir showed very slow dissolution in the dissolution medium selected to create and maintain a sink condition for the drug. The dissolution medium comprising 0.4% Tween 80 in water was predetermined to provide adequate solubility for the dose of Remdesivir selected for the dissolution test. Sink conditions ensured that there was sufficient solubility of Remdesivir in the medium to generate a concentration gradient between the saturated solution at the surface of a dissolving drug particle and the bulk of the dissolution medium so as to drive the diffusion of the drug molecules away from the dissolving particle-surface towards the bulk. Despite such sink conditions, the Remdesivir drug substance in the capsule (FIG. 2; “Remdesivir in Cap”) performed much more poorly compared to the control Acetaminophen (FIG. 2; “Acetaminophen API in Cap”), which has high oral bioavailability. For oral formulations of drugs with very poor solubility, such as Remdesivir, a slow-rate of dissolution would reduce the rate of absorption and rob the precious window of time available for absorption from the intestine, resulting in lower oral bioavailability.

In contrast, Example formulation 1 (ES040-36) both added directly as a solution (see FIG. 2, diamond datapoint with dashed line, “ES050-36 Without Capsule”) and when filled into a hard-gelatin capsule (see FIG. 2, diamond datapoint with solid line, “ES050-36 in Capsule”) significantly improved the rate of dissolution and rapidly reached maximum concentration of Remdesivir in the dissolution medium.

The above results demonstrate that the pharmaceutical compositions described herein can increase the solubility and dissolution rate of Remdesivir compared to the API in capsule and that with added SBEβCD—the solubilizing excipient used in currently marketed formulations. As a result, the pharmaceutical compositions described herein may be suitable for oral administration and have improved absorption in the gastrointestinal tract as compared Remdesivir alone or that in presence of SBEβCD, thereby leading to improved bioavailability. Furthermore, in the pharmaceutical compositions described herein, Remdesivir can also be easily diluted in aqueous media without the risk of precipitation, thereby, providing a strategy of administering the drug as an injection, an infusate, or as an oral solution at various concentrations to allow for accurate dosing without the need for large quantities of the solubilizing excipient, SBEβCD, that is contraindicated for very young children and in adults with impaired kidney functions.

### In Vivo Beagle Study

An in vivo dog study in beagles was carried out to determine the pharmacokinetic (PK) profile of Remdesivir following 1) a single intravenous (IV) infusion of a control formulation and 2) an oral (PO) dose of an Example pharmaceutical composition ES040-72 according to the present invention.

TABLE 10
Pharmaceutical composition used in beagle study - ES040-72

| Ingredient | Weight (g) | % (w/w) |
|---|---|---|
| Remdesivir | 2.02 | 6.7 |
| Polyethylene Glycol | 15.7721 | 52.34 |
| 400 (PEG 400) | | |
| Polyethylene Glycol | 4.7435 | 15.74 |
| 300 (PEG 300) | | |
| Cysteine Hydrochloride | 1.7370 | 5.76 |
| Monohydrate, USP | | |
| N-Acetyl-L-Cysteine | 1.7744 | 5.89 |
| Tween 80-NV-LQ-(AP) | 1.7227 | 5.72 |
| Ethyl Alcohol, 190 | 1.0244 | 3.40 |
| Proof, USP | | |
| Benzyl Alcohol, BP, | 1.3371 | 4.44 |
| NF, Ph. Eur | | |
| Total Weight | 30.1312 | |

TABLE 11
Test System for the in vivo study

| | |
|---|---|
| Strain/species/sex | Dog (Canis familiaris)/Beagle/Male |
| Source | Testing facility stock colony; originally procured |
| | from Marshall BioResources, North Rose, NY. |
| Identification | tattoo with matching cage card |
| Number of | 3 Males |
| Animals | |
| Age Range | Between 10 months to 3 years of age |
| Weight Range | Approximately 9-11 kg |
| History | Animals were experimentally non-naïve. |
| Housing/Sanitation | The animals were housed individually |
| | in species appropriate caging. |
| Diet | Certified canine diet was provided daily in amounts |
| | appropriate for the size and age of the animals. |
| Water | Tap water was available ad libitum. |
| Fasting | Animals were fasted overnight and had their daily |
| | ration offered 4 hours post-dose after the 4 hour |
| | sample collection. |
| Environment | Temperature: 64-84° F. (18-29° C.) |
| | Humidity: 30-70% |
| | Light: An alternating 12-hour light/dark cycle was |
| | maintained, except during study-specific procedures |
| | Ventilation: The airflow was set for at least 10 air |
| | changes per hour with 100% fresh air (no air |
| | recirculation). |

TABLE 12
Dosage of Control and Example Formulations

| | Number | | Dose | Dose | Dose |
|---|---|---|---|---|---|
| | of Dogs | Dose | Level | Conc | Volume |
| Test Article | (Male) | Route | (mg/kg) | (mg/mL) | (mL/kg) |
| Control^{a} | 3 | IV^{a} | 20 | 2^{a} | 10 |
| Example | 3 | Oral | 20 | ca. 50 | ca. 0.4 |
| Pharmaceutical | | (PO)^{b} | | to 60^{b} | to 0.333^{b} |
| Composition | | | | | |
| ES040-72^{b} | | | | | |

^{a}The Remdesivir Control Formulation (a 5 mg/mL solution) was matching to VEKLURY ® (i.e. a commercially available Remdesivir formulation). This was diluted with sterile water-for injection (WFI) to 2 mg/mL. The appropriate dose (by weight) was administered by a single 30 min infusion via cerphalic vein using Medfusion 2001 syringe pumps.
^{b}The Example Pharmaceutical Composition ES040-72 was placed in a Torpac hard gelatin capsule size # 12 (i.e. 5 mL volume) for dosing. The appropriate dose volume was calculated based on the exact dose concentration. The dogs received a single Torpac capsule. Following capsule administration, a squirt of water was given orally to aid transfer of the capsule to the stomach.

### Plasma Collection

For both IV or oral (PO) administered doses, plasma samples were collected over a period of 48 hours. Further details of the plasma collection are detailed in Table 13.

TABLE 13
Blood Sample Collection for Plasma

| | |
|---|---|
| Sampling | Jugular vein or alternative non-dosed vein |
| Location | |
| Blood Sample | ~1 mL |
| Volume | |
| Time Point | Protease inhibitor cocktail (see preparation instructions |
| of Plasma | outlined in footnote) equivalent to 5% of the blood |
| | volume (i.e. 50 μL added to 1 mL of blood) was added |
| | to the blood collection tubes to stabilize all analytes. |
| | Blood was collected from each animal in the IV |
| | group at pre-dose, 5 min (during infusion), 15 min |
| | (during infusion), 30 min (end of infusion), 35 min, 45 |
| | min, 1 h, 2 h, 12 h, 24 h, and 48 h from start of the |
| | infusion. The time-points are relative to the beginning |
| | of infusion. |
| | Blood was collected from each animal in the PO |
| | group at pre-dose, 0.25, 0.5, 1, 2, 4, 6, 8, 12, 24, and |
| | 48 hours post-dose. |
| Whole Blood | Samples were inverted several times following |
| Conditions | collection and held on wet ice until centrifuged. |
| Anti-coagulant | K3EDTA |
| Separation | Samples were centrifuged within 30 minutes of |
| Method | collection under refrigeration (set at 5° C. for |
| | 10 minutes at 2000 g). Plasma samples were stored |
| | in a freezer set to maintain ≤−70° C. |
| | until analyzed. |
| Bioanalysis | LC-MS/MS analysis of plasma samples was |
| | performed using Frontage Tier 2 non-GLP |
| | bioanalytical assay. Plasma was analyzed |
| | for (GS-441524), i.e. Compound (C). |
| Pharmacokinetic | The concentrations of the analytes determined by |
| Analysis | LC/MS/MS method was used to determine the |
| | pharmacokinetic parameters by Phoenix ® |
| | WinNonlin ® software (version 8.1, Certara, |
| | Princeton, NJ) using non-compartmental analyses. |

Protease cocktail Instructions: a. Add 1 mL of KF Solution (4 mg/mL) [prepared by weighing 80 mg of KF and mixing with 20 mL Ultrapure deionized water], b. Dissolve 1 tablet of Complete Tablet (Mini EDTA free Easypack protease inhibitor cocktail tablet, Roche, Cat. No 04 693 159 001), c. Mix thoroughly until tablet has dissolved, d. Add 1 mL of 25 mM EDTA Solution in water (the pH adjusted to 7 using 1N potassium hydroxide), e. Mix thoroughly, f. Store at 2-8° C.

### PBMC (Peripheral Blood Mononuclear Cells) Collection

For both IV or oral (PO) administered doses, blood samples were collected to determine the presence of analytes in PBMCs. Further details are detailed in Table 14.

TABLE 14
PBMC Collection

| | |
|---|---|
| Sampling | Jugular vein or alternative non-dosed vein |
| Location | |
| Blood Sample | ~4 mL |
| Volume | |
| Sample | Protease inhibitor cocktail (see preparation instructions |
| Collection | outlined in footnote above) equivalent to 5% of the blood |
| | volume (i.e. 200 μL added to 4 mL of blood) was added |
| | to the blood collection tubes to stabilize all analytes. |
| | Blood was collected at multiple time-points post-dose |
| | into cell preparation tubes. |
| Whole | Cell preparation tubes were stored in wet ice following |
| Blood | blood collection and processed within 2 hours of |
| Conditions | collection. Tubes were inverted several times prior |
| | to centrifugation. |
| Anti-coagulant | Sodium citrate |
| Separation | 1. Centrifuge CPT tubes in a horizontal rotor (swing |
| Method | out head) set at 18° C., 30 min, at 1700 × g. |
| | 2. After centrifugation, gently invert the unopened |
| | tube 5-10 times to re-suspend cells in plasma. To |
| | collect cells, uncap the tube and transfer entire |
| | contents of the tube above the gel, using a |
| | disposable transfer pipette, to a separate 15-mL |
| | conical tube. |
| | 3. Add PBS (Phosphate Buffered Saline) to bring |
| | volume to 15-mL graduated conical tube line and |
| | mix by inverting 5 times. Centrifuge tubes set at: |
| | 18° C., 15 min, at 300 × g. |
| | 4. Using a disposable transfer pipette, aspirate and |
| | dispose as much supernatant as possible without |
| | disturbing the cell pellet. |
| | 5. Re-suspend cell pellet by gently vortexing |
| | 6. Add 10 mL PBS to the tube using a disposable |
| | graduated pipette, and mix cells by inverting 5 |
| | times. Centrifuge set at: 18° C., 10 min, |
| | at 300 × g. |
| | 7. Using a disposable transfer pipette, aspirate and |
| | dispose as much supernatant as possible without |
| | disturbing the cell pellet. |
| | 8. In the same tube, snap-freeze the cell pellet in |
| | liquid nitrogen and store in a freezer set to |
| | maintain <−70° C. |
| Bioanalysis | LC-MS/MS analysis of plasma samples was |
| | performed using Frontage Tier 2 non-GLP |
| | bioanalytical assay. Plasma was analyzed for |
| | (GS-441524), i.e. Compound (C). |

### Results

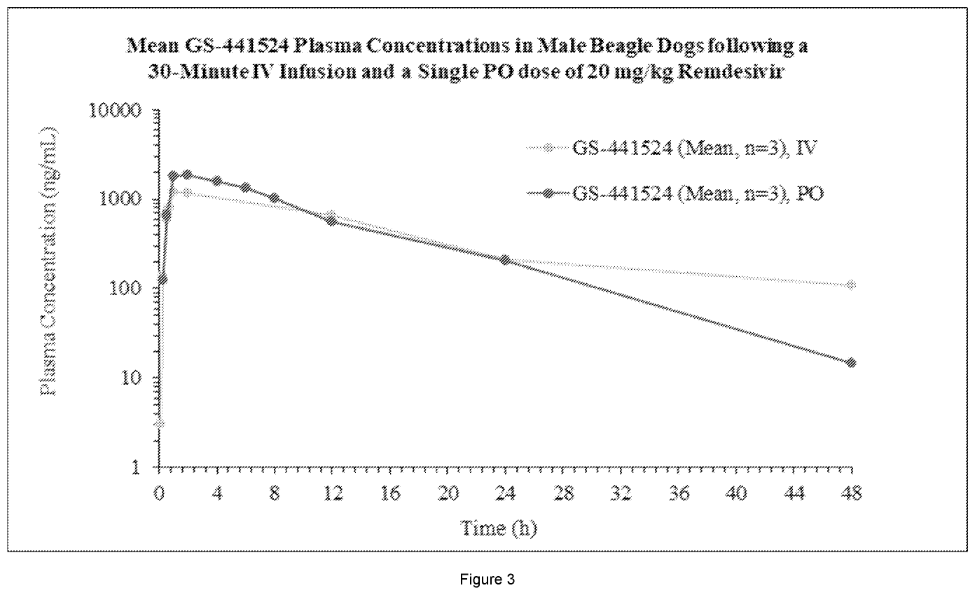

As indicated by FIG. 3 and as demonstrated by Table 15, the oral dosage form successfully matches the systemic exposure of the key active metabolite GS-441524 between both the IV and oral administration routes. Since this same key metabolite is detected in the blood plasma following IV administration and at levels that are sustained well above those of the prodrug, Remdesivir, as reported in primate and human studies, there is evidence to suggest that GS-441524 is taken up into the target tissue and converted to the active triphosphate metabolite following phosphorylation. GS-441524 has already been shown in the literature to show considerable in vitro activity against various SARS-CoV-2 infected cell lines and the plasma concentrations of this metabolite following IV administration in vivo are consistently above its own EC50, while that of Remdesivir rapidly declines to well below its own EC50. As a result, matching exposure and plasma levels of the key metabolite following oral administration of Remdesivir, are expected to demonstrate comparable pharmacological activity as the IV infusion.

The present pharmaceutical formulation and strategy of administering Remdesivir, instead of GS-441524, are nevertheless important to achieve maximum concentration and exposure of GS-441524.0n its own, GS-441524, has poor oral bioavailability due to poor permeability through the intestinal membrane.

TABLE 15
Plasma Pharmacokinetic Summary for GS-441524, i.e. Compound (C)

| | Nominal | AUCₗₐₛₜ | AUC_{inf} | t_{1/2} | tₘₐₓ | Cₘₐₓ |
|---|---|---|---|---|---|---|
| Administration | Dose | (h*ng/mL) | (h*ng/mL) | (h) | (h) | (ng/mL) |
| IV | 20 mg/kg | 19800 ± 2510 | 22000 ± 1950 | 15.6 ± 2.92 | 1.67 ± 0.577 | 1240 ± 203 |
| Oral | 20 mg/kg | 21500 ± 2100 | 21700 ± 2170 | 6.81 ± 0.270 | 1.33 ± 0.577 | 1980 ± 270 |
| (PO) | | | | | | |

In addition, GS-441524 (Compound (C) was detected in PBMC cells following both IV and oral (PO) administration.

### In Vitro Metabolism Study

The Relative abundance (%) of metabolites post-incubation of Remdesivir (10 μM) was determined after culture with human hepatocytes (1.3 million cells/mL). The results are shown in Table 16.

Samples were analysed by ultra-high performance liquid chromatography tandem mass spectrometry (UHPLC-MS/MS) with a quadrople time of flight mass spectrometer, for acquisition of high-resolution accurate mass data, with electrospray ionization incorporating in-line UV detection with a photodiode array detector.

TABLE 16
Relative abundance of analytes post-incubation with Remdesivir

| Component | 2 h | 6 h | 24 h |
|---|---|---|---|
| Remdesivir | 4.8 | 0.1 | 0.7 |
| Compound (A) | | | |
| GS-704277 (alanine | 0.0 | 2.4 | 2.2 |
| metabolite) | | | |
| Compound (B) | | | |
| Nucleoside | ND | 24.8 | 13.4 |
| monophosphate | | | |
| (RT 1.32 min) | | | |
| Nucleoside | 44.6 | 7.5 | 3.6 |
| monophosphate | | | |
| (RT 2.15 min) | | | |
| GS-441524 | 50.6 | 65.1 | 80.1 |
| Compound (C) | | | |
| Cyclic anhydride | ND | ND | ND |
| Nucleoside | ND | ND | ND |
| triphosphate | | | |

ND—not detected

These results demonstrate that it is expected that the predominant end product of 1^{st} pass metabolism of Remdesivir by liver to be GS-441524, which is the same metabolite that is the predominant and sustained species in the blood plasma following intravenous (IV) administration of Remdesivir. Following IV administration, Remdesivir is converted to GS-441524 by the esterases present in the blood. Hence, these in vitro results suggest that the exposure to GS-441524 is the same as that following IV administration.

1. A pharmaceutical composition comprising a compound of Formula (I) 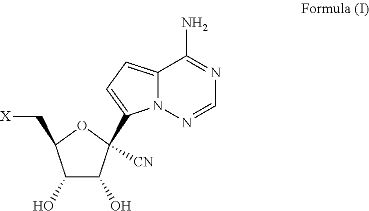
or a pharmaceutically acceptable salt thereof;
wherein X is selected from a hydroxyl, a metal salt hydroxylate, an O-linked phosphoester, an O-linked phosphoramidite, an O-linked ester, an O-linked carbamate, an S-linked phosphothioate, or an N-linked phosphoramidite, and
at least one pharmaceutically acceptable excipient selected from a cysteine compound, an amino acid, an amino acid salt, an N-acetyl amino acid, an acid or a salt thereof, or any combination thereof.

2. The pharmaceutical composition according to claim 1, wherein the pharmaceutical composition is free of cyclodextrin.

3. The pharmaceutical composition according to claim 1, wherein the at least one pharmaceutically acceptable excipient comprises at least one cysteine compound.

4. The pharmaceutical composition according to claim 3, wherein the at least one cysteine compound is cysteine hydrochloride and/or N-acetyl cysteine.

5. The pharmaceutical composition according to claim 1, wherein the pharmaceutical composition is a liquid formulation and wherein the pharmaceutical composition comprises one or more co-solvents.

6. The pharmaceutical composition according to claim 5, wherein the one or more co-solvents is selected from PEG, benzyl alcohol, ethanol or a combination thereof.

7. The pharmaceutical composition according to claim 5 comprising
3-10% w/w of compound of Formula (I)
0.5-30% w/w of a cysteine compound
50-86% w/w of one or more co-solvent.

8. The pharmaceutical composition according to claim 1, comprising one or more surfactants

9. The pharmaceutical composition according to claim 8, wherein the one or more surfactants comprises polysorbate.

10. The pharmaceutical composition according to claim 8, comprising
3-10% w/w of compound of Formula (I)
1-15 w/w cysteine hydrochloride monohydrate
3-15% w/w N-acetyl cysteine
50-83% w/w one or more co-solvent
2-8% w/w surfactant.

11. The pharmaceutical composition according to claim 1, wherein the compound of Formula (I) is

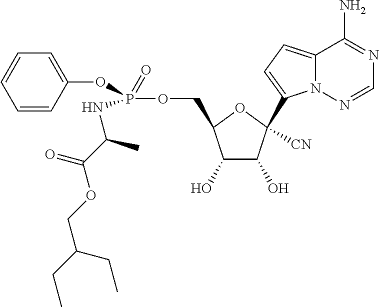 [SMILES: CCC(CC)COC(=O)[C@H](C)N[P@](=O)(OC[C@H]1O[C@@](C#N)(c2ccc3c(N)ncnn23)[C@H](O)[C@@H]1O)Oc1ccccc1]

12. A method of treating a viral infection, the method comprising administering to a subject in need thereof a therapeutically effective amount of the pharmaceutical composition of claim 1.

13. The method according to claim 11, wherein the virus causing the viral infection is selected from a coronavirus, respiratory syncytial virus, ebola, hepatitis, junin, lassa fever, orthomyxovirus, Hepatitis Virus (HV) type, disease-causing picornavirus, Ebola, SARS, MERS, respiratory syncytial virus and other pneumovirus, influenza, polio measles and retrovirus including adult Human T-cell lymphotropic virus type 1 (HTLV-1) and human immunodeficiency virus (HIV).

14. The method according to claim 12, wherein the pharmaceutical composition is administered orally or parenterally.

15. A capsule comprising the pharmaceutical composition of claim 1.

16. An oral solution comprising the pharmaceutical composition of claim 1.

17. An injectable solution comprising the pharmaceutical composition of claim 1.
